(19) United States
(12) Patent Application Publication
(10) Pub. No.: US 2026/0076912 A1
(43) Pub. Date: Mar. 19, 2026

(54) PHARMACEUTICAL COMPOSITIONS AND METHODS

(71) Applicant: Douglas Pharmaceuticals Ltd, Lincoln (NZ)

(72) Inventors: Peter William Surman, Lincoln (NZ); Zhen Shi, Lincoln (NZ); Fergus Cameron Binnie, Lincoln (NZ)

(21) Appl. No.: 19/326,549

(22) Filed: Sep. 11, 2025

Related U.S. Application Data
(60) Provisional application No. 63/694,671, filed on Sep. 13, 2024.

(51) Int. Cl. A61K 9/20; A61K 9/28; A61K 31/135

(52) U.S. Cl. CPC A61K 9/2054; A61K 9/2013; A61K 9/2027; A61K 9/282; A61K 9/2893; A61K 31/135

(57) ABSTRACT

Described is an extended release pharmaceutical composition. The composition comprises a therapeutically effective amount of an active agent selected from the group consisting of ketamine, norketamine, pharmaceutically acceptable salts thereof, and combinations thereof. The active agent is incorporated into a sustained-release polymeric matrix. The matrix comprises a matrix polymer, and a permeability-modifying filler. A method of preventing, treating and/or managing treatment-resistant depression in a subject is also or alternatively describe.

### RELATED APPLICATIONS

This application claims priority under 35 U.S.C. 119 (e) to U.S. Provisional Patent Application Ser. No. 63/694,671, entitled “Pharmaceutical Compositions and Methods”, filed Sep. 13, 2024, the disclosure of which is hereby incorporated by reference in its entirety.

### BACKGROUND

Ketamine has been used for anesthesia since the 1960s. In the United States ketamine injection has been approved by the FDA (U.S. Food and Drug Administration) for use as a general anesthetic (for example Endo's Ketalar® injection). More recently esketamine hydrochloride (the S enantiomer of ketamine) has been registered as a nasal spray to be used in conjunction with an oral antidepressant for the treatment of treatment-resistant depression (TRD) in adults and depressive symptoms in adults with major depressive disorder (MDD) with acute suicidal ideation or behavior. However, Ketamine is also abused as a recreational drug for its hallucinogenic and dissociative effects.

There is a risk with some pharmaceuticals, such as opioids and ketamine, that products intended for medicinal purposes may be abused. Further, products may be abused via administration routes that the product is not intended or formulated for, for example tablets may be crushed and snorted, or crushed, dissolved and injected. Opioids have been reformulated with abuse deterrent formulations (ADFs) to make them more difficult to crush and dissolve. For example, some ADF's include use of high-molecular weight (HMW) polyethylene oxide (PEO). However, post marketing data relating to the reformulated ADFs (for example OxyContin® analgesic by Purdue Pharma and Opana® ER by Endo Pharmaceuticals) that were reformulated with PEO showed that while abuse was decreased, particularly via snorting, where abusers were able to crush, dissolve and inject the ADF tablets, it resulted in unexpected consequences, such as cases of a potentially fatal bleeding disorder resembling thrombotic thrombocytopenic purpura (TTP), and renal failure with thrombotic microangiopathy (TMA) observed in the kidneys. It is thought that these conditions may be due, at least in part, to undissolved particles of PEO in the injected solution. Opana ER has since been withdrawn from the market at least in part due to the unintended side-effects of the reformulation with PEO.

WO2019073408, WO2020194087 and WO2022064387 (all Douglas Pharmaceuticals Limited) describe an extended release formulation and dosing regimen for the treatment of treatment-resistant depression, treatment-resistant anxiety, and phobia. WO2019073408, WO2020194087 and WO2022064387 describe extended release formulations of ketamine.

The results of the BEDROC study sponsored by Douglas Pharmaceuticals Limited were published in Nature Medicine (Glue et al. “Extended-release ketamine tablets for treatment-resistant depression: a randomized placebo-controlled phase 2 trial”, Nature Medicine, Volume 30, July 2024, 2004-2009). In this study, 231 patients with treatment-resistant depression (TRD) were treated with R-107, an extended release formulation of ketamine including PEO, 120 mg per day for 5 days, and 168 (72.7%) patients were included as an enriched responder population who were randomized to a range of double-blind R-107 doses or placebo for the next 12 weeks. In this double-blind phase, the 180 mg dose given twice weekly showed statistically significant and clinically meaningful improvement in depressive symptoms based on MADRS score compared with placebo, with a group-treatment difference of 6.1. The pharmacokinetics, including the release profile, of the dosage course were shown to be the critical and surprising advantages of the study.

It would be beneficial to have a treatment/composition that provides the same or substantially equivalent pharmacokinetics as the previously studied dosage course, including R-107, without using a composition based on PEO.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY

In a first aspect there is provided an extended release pharmaceutical composition comprising:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
a matrix polymer, and
a permeability-modifying filler.

In a second aspect there is provided an extended release pharmaceutical composition for prevention, treatment and/or management, of a subject in need thereof comprising:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
a matrix polymer, and
a permeability-modifying filler.

In one aspect there is provided a method of preventing, treating and/or managing depression, anxiety, alcohol abuse disorder, and/or pain in a subject, the method comprising administering an extended release pharmaceutical composition as described in the first or second aspects.

In one aspect there is provided a method of preventing, treating and/or managing depression in a subject, the method comprising administering an extended release pharmaceutical composition as described in the first or second aspects.

In one aspect there is provided a method of preventing, treating and/or managing treatment-resistant depression in a subject, the method comprising administering an extended release pharmaceutical composition as described in the first or second aspects.

In one aspect there is provided use of an extended release pharmaceutical composition as described in the first or second aspects in the manufacture of a medicament for prevention, treatment and/or management of depression, anxiety, alcohol abuse disorder, and/or pain.

In one aspect there is provided use of an extended release pharmaceutical composition as described in the first or second aspects in the manufacture of a medicament for prevention, treatment, and/or management of depression.

In one aspect there is provided use of an extended release pharmaceutical composition as described in the first or second aspects in the manufacture of a medicament for prevention, treatment, and/or management of treatment-resistant depression.

In one aspect there is provided an extended release pharmaceutical composition as described in the first or second aspects for prevention, treatment, and/or management of depression, anxiety, alcohol abuse disorder, and/or pain.

In one aspect there is provided an extended release pharmaceutical composition as described in the first or second aspects for prevention, treatment, and/or management of depression.

In one aspect there is provided an extended release pharmaceutical composition as described in the first or second aspects for prevention, treatment, and/or management of treatment-resistant depression.

In one aspect there is provided use of an extended release pharmaceutical composition as described in the first or second aspects for prevention, treatment, and/or management of depression, anxiety, alcohol abuse disorder, and/or pain.

In one aspect there is provided use of an extended release pharmaceutical composition as described in the first or second aspects for prevention, treatment, and/or management of depression.

In one aspect there is provided use of an extended release pharmaceutical composition as described in the first or second aspects for prevention, treatment, and/or management of treatment-resistant depression.

In one aspect there is provided a method of manufacturing an extended release pharmaceutical composition, the method comprising: blending a therapeutically effective amount of an active agent selected from the group consisting of ketamine, norketamine, pharmaceutically acceptable salts thereof, and combinations thereof, with a matrix polymer and a permeability-modifying filler

In at least one example, the depression is selected from treatment-resistant depression, major depressive disorder (MDD) (for example, without limitation, with acute suicidal ideation or behavior), and bipolar depression.

In at least one example, the depression is treatment resistant depression.

In at least one example, the anxiety includes treatment-resistant anxiety.

In at least one example, the anxiety DSM-V Generalized Anxiety Disorder (GAD), Social Anxiety Disorder, Panic Disorder, Post-Traumatic Stress Disorder (PTSD), and/or Obsessive-Compulsive Disorder.

In at least one example, the active agent is ketamine. In at least one example, the active agent is racemic mixture of (R)-ketamine and (S)-ketamine. In at least one example, the active agent is ketamine hydrochloride.

In at least one example, the extended release pharmaceutical composition comprises about 10 to 60% w/w of the active agent. In at least one example, the extended release pharmaceutical composition comprises about 12 to 50% w/w of the active agent. In at least one example, the extended release pharmaceutical composition comprises about 12 to 40% w/w of the active agent. In at least one example, the extended release pharmaceutical composition comprises about 12 to 30% w/w of the active agent. In at least one example, the extended release pharmaceutical composition comprises about 12 to 25% w/w of the active agent. In at least one example, the extended release pharmaceutical composition comprises about 12 to 20% w/w of the active agent. In at least one example, the extended release pharmaceutical composition comprises about 12 to 18% w/w of the active agent.

In at least one example, the extended release pharmaceutical composition comprises about 10 to 60% w/w ketamine. In at least one example, the extended release pharmaceutical composition comprises about 12 to 50% w/w ketamine. In at least one example, the extended release pharmaceutical composition comprises about 12 to 40% w/w ketamine. In at least one example, the extended release pharmaceutical composition comprises about 12 to 30% w/w ketamine. In at least one example, the extended release pharmaceutical composition comprises about 12 to 25% w/w ketamine. In at least one example, the extended release pharmaceutical composition comprises about 12 to 20% w/w ketamine. In at least one example, the extended release pharmaceutical composition comprises about 12 to 18% w/w ketamine.

In at least one example, the extended release pharmaceutical composition comprises about 10 to 25% w/w ketamine hydrochloride. In at least one example, the extended release pharmaceutical composition comprises about 12 to 20% w/w ketamine hydrochloride. In at least one example, the extended release pharmaceutical composition comprises about 15 to 20% w/w ketamine hydrochloride.

In at least one example, the extended release pharmaceutical composition comprises about 20 to 78% w/w of the matrix polymer. In at least one example, the extended release pharmaceutical composition comprises about 25 to 78% w/w of the matrix polymer. In at least one example, the extended release pharmaceutical composition comprises about 35 to 78% w/w of the matrix polymer. In at least one example, the extended release pharmaceutical composition comprises about 40 to 78% w/w of the matrix polymer.

In at least one example, the matrix polymer forms a hydrogel in aqueous conditions.

In at least one example, the matrix polymer has a viscosity of equal to or greater than about 2,000 mPa·s, or equal to or greater than about 3,000 mPa·s, or equal to or greater than about 4,000 mPa·s (1% w/v in water at 20° C.).

In at least one example, the matrix polymer has a viscosity of about 2,000 to 150,000 mPa·s (1% w/v in water at 20° C.). In at least one example, the matrix polymer has a viscosity of about 3,000 to 150,000 mPa·s (1% w/v in water at 20° C.). In at least one example, the matrix polymer has a viscosity of about 4,000 to 150,000 mPa·s (1% w/v in water at 20° C.).

In at least one example, the matrix polymer comprises carbomer homopolymer with a viscosity greater than about 29,400 mPa·s (measured at 0.5% w/v in water at 25° C.). In at least one example, the matrix polymer comprises carbomer homopolymer with a viscosity range of about 29,400 to 60,000 mPa·s (measured at 0.5% w/v in water at 25° C.).

In at least one example, the matrix polymer comprises hydroxypropyl methylcellulose with a viscosity of greater than about 4,000 mPa·s (2% w/v in water at 20° C.). In at least one example, the matrix polymer comprises hydroxypropyl methylcellulose with a viscosity of greater than about 8,000 mPa·s (2% w/v in water at 20° C.). In at least one example, the matrix polymer comprises hydroxypropyl methylcellulose with a viscosity range of about 4,000 to 300,000 mPa·s (2% w/v in water at 20° C.). In at least one example, the matrix polymer comprises hydroxypropyl methylcellulose with a viscosity range of about 8,000 to 300,000 mPa·s (2% w/v in water at 20° C.). In at least one example, the matrix polymer comprises hydroxypropyl methylcellulose with a viscosity range of about 8,000 to 150,000 mPa·s (2% w/v in water at 20° C.).

In at least one example, the matrix polymer is selected from one or more of the group hydroxypropyl methylcellulose, a carbomer homopolymer, and hydroxypropylcellulose.

In at least one example, the carbomer homopolymer is carbomer homopolymer Type B and/or carbomer homopolymer Type C.

In at least one example, the matrix polymer comprises hydroxypropyl methylcellulose (HPMC).

In at least one example, the matrix polymer is hydroxypropyl methylcellulose (HPMC).

In at least one example, the HPMC is selected from one or more of the group HPMC K100M, HPMC K4M, HPMC K15M, HPMC K200M, and HPMC K35M.

In at least one example, the composition does not include polyethylene oxide (PEO). In at least one example, the composition does not include polyethylene oxide (PEO) of molecular weight of from about 2 million to about 7 million.

In at least one example, the ratio of the active agent to the matrix polymer is about 1:1.5 to 1:4.

In at least one example, the extended release pharmaceutical composition comprises about 3 to 60% w/w of the permeability-modifying filler.

In at least one example, the permeability-modifying filler comprises microcrystalline cellulose, starch (for example, without limitation, corn starch, pea starch, potato starch, rice starch, tapioca starch, wheat starch, arrowroot starch), pregelatinized starch, mannitol, polyvinylpyrrolidone, lactose (for example, without limitation, lactose monohydrate), fructose, dextrose, sorbitol, sucrose, maltodextrin, and/or calcium salts (for example, without limitation, calcium carbonate, calcium phosphate-dibasic, calcium phosphate-tribasic, calcium sulfate).

In at least one example, the permeability-modifying filler is selected from one or more of the group microcrystalline cellulose (MCC), starch (for example, without limitation, corn starch, pea starch, potato starch, rice starch, tapioca starch, wheat starch, arrowroot starch), pregelatinized starch, mannitol, polyvinylpyrrolidone, lactose (for example, without limitation, lactose monohydrate), fructose, dextrose, sorbitol, sucrose, maltodextrin and calcium salts (for example, without limitation, calcium carbonate, calcium phosphate-dibasic, calcium phosphate-tribasic, calcium sulfate).

In at least one example, the permeability-modifying filler comprises microcrystalline cellulose.

In at least one example, the extended release pharmaceutical composition comprises about 20 to 60% w/w of the permeability-modifying filler, wherein the permeability-modifying filler is slightly soluble, very slightly soluble, practically insoluble, or insoluble in water at about 20° C.

In at least one example, the extended release pharmaceutical composition comprises about 20 to 60% w/w of the permeability-modifying filler, wherein the permeability-modifying filler is practically insoluble, or insoluble in water at about 20° C.

In at least one example, the extended release pharmaceutical composition comprises about 25 to 60% w/w of the permeability-modifying filler, wherein the permeability-modifying filler is practically insoluble, or insoluble in water at about 20° C.

In at least one example, the extended release pharmaceutical composition comprises about 25 to 55% w/w of the permeability-modifying filler, wherein the permeability-modifying filler is practically insoluble, or insoluble in water at about 20° C.

In at least one example, the permeability-modifying filler that is slightly soluble, very slightly soluble, practically insoluble, or insoluble in water at about 20° C. is selected from one or more of microcrystalline cellulose, starch (for example, without limitation, corn starch, pea starch, potato starch, rice starch, tapioca starch, wheat starch, arrowroot starch), calcium salts (for example, without limitation, calcium carbonate, calcium phosphate-dibasic, calcium phosphate-tribasic, calcium sulfate), and pregelatinized starch.

In at least one example, the permeability-modifying filler that is practically insoluble or insoluble in water at about 20° C. is selected from one or more of microcrystalline cellulose, starch (for example, without limitation, corn starch, pea starch, potato starch, rice starch, tapioca starch, wheat starch, arrowroot starch), calcium carbonate, and dicalcium phosphate.

In at least one example, the composition comprises about 25 to 55% w/w of the permeability-modifying filler, wherein the permeability-modifying filler is microcrystalline cellulose.

In at least one example, the extended release pharmaceutical composition comprises about 3 to 35% w/w of the permeability-modifying filler, wherein the permeability-modifying filler is very soluble or freely soluble in water at about 20° C.

In at least one example, the extended release pharmaceutical composition comprises about 3 to 30% w/w of the permeability-modifying filler, wherein the permeability-modifying filler is very soluble or freely soluble in water at about 20° C.

In at least one example, the extended release pharmaceutical composition comprises about 3 to 20% w/w of the permeability-modifying filler, wherein the permeability-modifying filler is very soluble or freely soluble in water at about 20° C.

In at least one example, the extended release pharmaceutical composition comprises about 3 to 15% w/w of the permeability-modifying filler, wherein the permeability-modifying filler is very soluble or freely soluble in water at about 20° C.

In at least one example, the permeability-modifying filler that is very soluble or freely soluble in water at about 20° C. is selected from one or more of lactose (for example, without limitation, lactose monohydrate), fructose, dextrose, sorbitol, sucrose, maltodextrin, mannitol, and polyvinylpyrrolidone.

In at least one example, the extended release pharmaceutical composition comprises about 3 to 20% w/w of the permeability-modifying filler, wherein the permeability-modifying filler is mannitol.

In at least one example, the extended release pharmaceutical composition comprises about 3 to 15% w/w of the permeability-modifying filler, wherein the permeability-modifying filler is mannitol.

In at least one example, the extended release pharmaceutical composition exhibits an in vitro release with at least one of the following:
(a) about 25 to 35% of the active substance is released at about the 1 hour time point,
(b) about 36 to 50% of the active substance is released at about the 2 hour time point,
(c) about 53 to 73% of the active substance is released at about the 4 hour time point,
(d) about 64 to 88% of the active substance is released at about the 6 hour time point,
(e) about 73 to 99% of the active substance is released at about the 8 hour time point,
(f) about 79 to 100% of the active substance is released at about the 10 hour time point,
(g) about 81 to 100% of the active substance is released at about the 12 hour time point,
   when tested according to the USP Apparatus 1 (basket) method at a rotation rate of 100 rpm in media at pH 1.0 at 37.0±0.5° C.

In at least one example, the extended release pharmaceutical composition exhibits an in vitro release with at least two of (a) to (g).

In at least one example, the extended release pharmaceutical composition exhibits an in vitro release with at least three of (a) to (g).

In at least one example, the extended release pharmaceutical composition exhibits an in vitro release with at least four of (a) to (g).

In at least one example, the extended release pharmaceutical composition exhibits an in vitro release with at least five of (a) to (g).

In at least one example, the extended release pharmaceutical composition exhibits an in vitro release of: about 36 to 50% of the active substance is released at about the 2 hour time point,
about 64 to 88% of the active substance is released at about the 6 hour time point,
about 81 to 100% of the active substance is released at about the 12 hour time point,
   when tested according to the USP Apparatus 1 (basket) method at a rotation rate of 100 rpm in media at pH 1.0 at 37.0±0.5° C.

In at least one example, the extended release pharmaceutical composition exhibits an in vitro release of:
about 36 to 50% of the active substance is released at about the 2 hour time point,
about 73 to 99% of the active substance is released at about the 8 hour time point,
about 81 to 100% of the active substance is released at about the 12 hour time point,
   when tested according to the USP Apparatus 1 (basket) method at a rotation rate of 100 rpm in media at pH 1.0 at 37.0±0.5° C.

In at least one example, the extended release pharmaceutical composition exhibits an in vitro release of:
about 41 to 50% of the active substance is released at about the 2 hour time point,
about 73 to 99% of the active substance is released at about the 8 hour time point,
about 81 to 100% of the active substance is released at about the 12 hour time point,
   when tested according to the USP Apparatus 1 (basket) method at a rotation rate of 100 rpm in media at pH 1.0 at 37.0±0.5° C.

In at least one example, the extended release pharmaceutical composition exhibits an in vitro release of about 48 to 60 mg of ketamine at 12 hours when tested according to the USP Apparatus 1 (basket) method at a rotation rate of 100 rpm in media at pH 1.0 at 37.0±0.5° C.

In at least one example, the extended release pharmaceutical composition exhibits an in vitro release of:
about 31 to 44 mg of ketamine at 4 hours, and
about 48 to 60 mg of ketamine at 12 hours,
   when tested according to the USP Apparatus 1 (basket) method at a rotation rate of 100 rpm in media at pH 1.0 at 37.0±0.5° C.,
wherein the extended release pharmaceutical composition is a tablet comprising about 60 mg of ketamine.

In at least one example, the extended release pharmaceutical composition exhibits an in vitro release of:
about 21 to 30 mg of ketamine at 2 hours,
about 31 to 44 mg of ketamine at 4 hours, and
about 48 to 60 mg of ketamine at 12 hours,
   when tested according to the USP Apparatus 1 (basket) method at a rotation rate of 100 rpm in media at pH 1.0 at 37.0±0.5° C.,
   wherein the extended release pharmaceutical composition is a tablet comprising about 60 mg of ketamine.

In at least one example, the extended release pharmaceutical composition exhibits an in vitro release of:
about 21 to 30 mg of ketamine at 2 hours,
about 38 to 52 mg of ketamine at 6 hours, and
about 48 to 60 mg of ketamine at 12 hours,
   when tested according to the USP Apparatus 1 (basket) method at a rotation rate of 100 rpm in media at pH 1.0 at 37.0±0.5° C.,
wherein the extended release pharmaceutical composition is a tablet comprising about 60 mg of ketamine.

In at least one example, the extended release pharmaceutical composition exhibits an in vitro release of:
about 59 to 80 mg of ketamine at 4 hours, and
about 92 to 120 mg of ketamine at 12 hours,
   when tested according to the USP Apparatus 1 (basket) method at a rotation rate of 100 rpm in media at pH 1.0 at 37.0±0.5° C.,
wherein the extended release pharmaceutical composition is a tablet comprising about 120 mg of ketamine.

In at least one example, the extended release pharmaceutical composition exhibits an in vitro release of:
about 40 to 55 mg of ketamine at 2 hours,
about 59 to 80 mg of ketamine at 4 hours, and
about 92 to 120 mg of ketamine at 12 hours,
   when tested according to the USP Apparatus 1 (basket) method at a rotation rate of 100 rpm in media at pH 1.0 at 37.0±0.5° C.,
wherein the extended release pharmaceutical composition is a tablet comprising about 120 mg of ketamine.

In at least one example, the extended release pharmaceutical composition exhibits an in vitro release of:
about 40 to 55 mg of ketamine at 2 hours,
about 72 to 98 mg of ketamine at 6 hours, and
about 92 to 120 mg of ketamine at 12 hours,
   when tested according to the USP Apparatus 1 (basket) method at a rotation rate of 100 rpm in media at pH 1.0 at 37.0±0.5° C.,
wherein the extended release pharmaceutical composition is a tablet comprising about 120 mg of ketamine.

In at least one example, the extended release pharmaceutical composition comprises about 30 to 240 mg of ketamine.

In at least one example, the extended release pharmaceutical composition comprises about 60 or 120 or 180 or 240 mg of ketamine.

In at least one example, the extended release pharmaceutical composition comprises about 60 or 120 mg of ketamine.

In at least one example, the extended release pharmaceutical composition comprises about 69 or 138 mg of ketamine hydrochloride.

In at least one example, the active agent is micronized.

In at least one example, the active agent has a particle size distribution D50 of less than about 500 μm. In at least one example, the active agent has a particle size distribution D50 of less than about 200 μm.

In at least one example, the active agent has a particle size distribution D50 of about 10 to 200 μm.

In at least one example, the extended release pharmaceutical composition is a tablet.

In at least one example, the extended release pharmaceutical composition is an oral tablet.

In at least one example, the oral tablet comprises about 30 to 240 mg of ketamine.

In at least one example, the oral tablet comprises about 50 to 80 mg of ketamine.

In at least one example, the oral tablet comprises about 100 to 140 mg of ketamine.

In at least one example, the oral tablet comprises about 30 mg, 60 mg, 120 mg, 180 mg, or 240 mg of ketamine.

In at least one example, the oral tablet comprises about 60 mg or 120 mg of ketamine.

In at least one example, the oral tablet comprises about 69 or 138 mg of ketamine hydrochloride.

In at least one example, the tablet has a mass of about 200 to 1000 mg. In at least one example, the tablet has a mass of about 300 to 850 mg. In at least one example, the tablet has a mass of about 300 to 600 mg. In at least one example, the tablet has a mass of about 300 to 500 mg. In at least one example, the tablet has a mass of about 700 to 1000 mg. In at least one example, the tablet has a mass of about 700 to 900 mg. In at least one example, the tablet has a mass of about 400 mg or 800 mg.

In at least one example, the tablet has a volume of about 0.2 to 1.2 mL. In at least one example, the tablet has a volume of about 0.4 to 0.6 mL. In at least one example, the tablet has a volume of about 0.8 to 1.2 mL.

In at least one example, the tablet has a mass of about 300 to 600 mg and comprises about 30 to 75% w/w of the matrix polymer.

In at least one example, the tablet has a mass of about 300 to 600 mg and comprises about 35 to 75% w/w of the matrix polymer.

In at least one example, the tablet has a mass of about 300 to 600 mg and comprises about 35 to 70% w/w of the matrix polymer.

In at least one example, the tablet has a mass of about 300 to 600 mg and comprises about 30 to 70% w/w of the matrix polymer.

In at least one example, the tablet has a mass of about 300 to 600 mg and comprises about 30 to 75% w/w of the matrix polymer, wherein the matrix polymer comprises HMPC.

In at least one example, the tablet has a mass of about 300 to 600 mg and comprises about 35 to 75% w/w of the matrix polymer, wherein the matrix polymer comprises HMPC.

In at least one example, the tablet has a mass of about 300 to 600 mg and comprises about 35 to 70% w/w of the matrix polymer, wherein the matrix polymer comprises HMPC.

In at least one example, the tablet has a mass of about 300 to 600 mg and comprises about 30 to 70% w/w of the matrix polymer, wherein the matrix polymer comprises HMPC.

In at least one example, the extended release pharmaceutical composition comprises about 30 to 75% w/w of the matrix polymer, wherein the extended release pharmaceutical composition is an oral tablet comprising about 30 to 60 mg of ketamine.

In at least one example, the extended release pharmaceutical composition comprises about 35 to 75% w/w of the matrix polymer, wherein the extended release pharmaceutical composition is an oral tablet comprising about 30 to 60 mg of ketamine.

In at least one example, the extended release pharmaceutical composition comprises about 35 to 70% w/w of the matrix polymer, wherein the extended release pharmaceutical composition is an oral tablet comprising about 30 to 60 mg of ketamine.

In at least one example, the tablet has a mass of about 700 to 1000 mg and comprises about 20 to 45% w/w of the matrix polymer.

In at least one example, the tablet has a mass of about 700 to 1000 mg and comprises about 25 to 45% w/w of the matrix polymer.

In at least one example, the tablet has a mass of about 700 to 1000 mg and comprises about 25 to 40% w/w of the matrix polymer.

In at least one example, the tablet has a mass of about 700 to 1000 mg and comprises about 20 to 45% w/w of the matrix polymer, wherein the matrix polymer comprises HMPC.

In at least one example, the tablet has a mass of about 700 to 1000 mg and comprises about 25 to 45% w/w of the matrix polymer, wherein the matrix polymer comprises HMPC.

In at least one example, the tablet has a mass of about 700 to 1000 mg and comprises about 25 to 40% w/w of the matrix polymer, wherein the matrix polymer comprises HMPC.

In at least one example, the extended release pharmaceutical composition comprises about 20 to 45% w/w of the matrix polymer, wherein the extended release pharmaceutical composition is an oral tablet comprising about 120 to 180 mg of ketamine.

In at least one example, the extended release pharmaceutical composition comprises about 25 to 45% w/w of the matrix polymer, wherein the extended release pharmaceutical composition is an oral tablet comprising about 120 to 180 mg of ketamine.

In at least one example, the extended release pharmaceutical composition comprises about 25 to 40% w/w of the matrix polymer, wherein the extended release pharmaceutical composition is an oral tablet comprising about 120 to 180 mg of ketamine.

In at least one example, wherein administration of the extended release pharmaceutical composition to a subject providing a dose of about 180 mg of ketamine results in a ketamine plasma concentration profile AUC of about 270 to 370 ng·hr/ml.

In at least one example, wherein administration of the extended release pharmaceutical composition to a subject providing a dose of about 180 mg of ketamine results in a ketamine plasma concentration profile AUC_{0-∞} of about 270 to 370 ng·hr/ml.

In at least one example, wherein administration of the extended release pharmaceutical composition to a subject providing a dose of about 180 mg of ketamine results in a ketamine plasma concentration profile AUC_{0-∞} of about 280 to 360 ng·hr/ml.

In at least one example, wherein administration of the extended release pharmaceutical composition to a subject providing a dose of about 180 mg of ketamine results in a ketamine plasma concentration profile AUC₀₋ₜ of about 270 to 370 ng·hr/ml.

In at least one example, wherein administration of the extended release pharmaceutical composition to a subject providing a dose of about 180 mg of ketamine results in a ketamine plasma concentration profile AUC₀₋ₜ of about 270 to 355 ng·hr/ml.

In at least one example, wherein administration of the extended release pharmaceutical composition to a subject providing a dose of about 180 mg of ketamine results in a norketamine plasma concentration profile AUC of about 3000 to 4000 ng·hr/ml.

In at least one example, wherein administration of the extended release pharmaceutical composition to a subject providing a dose of about 180 mg of ketamine results in a norketamine plasma concentration profile AUC_{0-∞} of about 3000 to 4000 ng·hr/ml.

In at least one example, wherein administration of the extended release pharmaceutical composition to a subject providing a dose of about 180 mg of ketamine results in a norketamine plasma concentration profile AUC_{0-∞} of about 3500 to 3900 ng·hr/ml.

In at least one example, wherein administration of the extended release pharmaceutical composition to a subject providing a dose of about 180 mg of ketamine results in a norketamine plasma concentration profile AUC₀₋ₜ of about 3000 to 4000 ng·hr/ml.

In at least one example, wherein administration of the extended release pharmaceutical composition to a subject providing a dose of about 180 mg of ketamine results in a norketamine plasma concentration profile AUC₀₋ₜ of about 3300 to 3800 ng·hr/ml.

In at least one example, wherein administration of the extended release pharmaceutical composition to a subject providing a dose of about 180 mg of ketamine results in a ketamine plasma concentration profile Cₘₐₓ of about 20 to 50 ng/ml.

In at least one example, wherein administration of the extended release pharmaceutical composition to a subject providing a dose of about 180 mg of ketamine results in a ketamine plasma concentration profile Cₘₐₓ of about 25 to 40 ng/ml.

In at least one example, wherein administration of the extended release pharmaceutical composition to a subject providing a dose of about 180 mg of ketamine results in a norketamine plasma concentration profile Cₘₐₓ of about 250 to 320 ng/ml.

In at least one example, wherein administration of the extended release pharmaceutical composition to a subject providing a dose of about 180 mg of ketamine results in a norketamine plasma concentration profile Cₘₐₓ of about 260 to 300 ng/ml.

In at least one example, wherein administration of the extended release pharmaceutical composition to a subject providing a dose of about 180 mg of ketamine results in a ketamine plasma concentration profile Tₘₐₓ of about 1.5 to 2.5 hr.

In at least one example, wherein administration of the extended release pharmaceutical composition to a subject providing a dose of about 180 mg of ketamine results in a ketamine plasma concentration profile Tₘₐₓ of about 1.5 to 2.4 hr.

In at least one example, wherein administration of the extended release pharmaceutical composition to a subject providing a dose of about 180 mg of ketamine results in a norketamine plasma concentration profile Tₘₐₓ of about 2 to 3 hr.

In at least one example, wherein administration of the extended release pharmaceutical composition to a subject providing a dose of about 180 mg of ketamine results in a norketamine plasma concentration profile Tₘₐₓ of about 2 to 2.9 hr.

In at least one example, wherein administration of the extended release pharmaceutical composition to a subject providing a dose of about 180 mg of ketamine results in a ketamine plasma concentration profile t_{1/2} of about 7 to 8 hr.

In at least one example, wherein administration of the extended release pharmaceutical composition to a subject providing a dose of about 180 mg of ketamine results in a ketamine plasma concentration profile t_{1/2} of about 7.2 to 7.8 hr.

In at least one example, wherein administration of the extended release pharmaceutical composition to a subject providing a dose of about 180 mg of ketamine results in a norketamine plasma concentration profile t_{1/2} of about 7.5 to 8.5 hr.

In at least one example, wherein administration of the extended release pharmaceutical composition to a subject providing a dose of about 180 mg of ketamine results in a norketamine plasma concentration profile t_{1/2} of about 7.8 to 8.1 hr.

In at least one example, wherein administration of the extended release pharmaceutical composition to a subject providing a dose of about 180 mg of ketamine results in a plasma concentration profile with a ratio of AUC norketamine/ketamine of about 8 to 15.

In at least one example, wherein administration of the extended release pharmaceutical composition to a subject providing a dose of about 180 mg of ketamine results in a plasma concentration profile with a ratio of AUC norketamine/ketamine of about 9 to 13.

In at least one example, wherein administration of the extended release pharmaceutical composition to a subject providing a dose of about 180 mg of ketamine results in a plasma concentration profile with a ratio of AUC_{0-∞} norketamine/ketamine of about 8 to 15.

In at least one example, wherein administration of the extended release pharmaceutical composition to a subject providing a dose of about 180 mg of ketamine results in a plasma concentration profile with a ratio of AUC_{0-∞} norketamine/ketamine of about 9 to 13.

In at least one example, wherein administration of the extended release pharmaceutical composition to a subject providing a dose of about 180 mg of ketamine results in a plasma concentration profile with a ratio of AUC₀₋ₜ norketamine/ketamine of about 8 to 15.

In at least one example, wherein administration of the extended release pharmaceutical composition to a subject providing a dose of about 180 mg of ketamine results in a plasma concentration profile with a ratio of AUC₀₋ₜ norketamine/ketamine of about 9 to 13.

In at least one example, wherein administration of the extended release pharmaceutical composition to a subject providing a dose of about 180 mg of ketamine results in a plasma concentration profile with a ratio of Cₘₐₓ norketamine/ketamine of about 6 to 10.

In at least one example, wherein administration of the extended release pharmaceutical composition to a subject providing a dose of about 180 mg of ketamine results in a plasma concentration profile with a ratio of Cₘₐₓ norketamine/ketamine of about 7 to 10.

In at least one example, the plasma concentration profile is based on untransformed data.

In at least one example, the plasma concentration profile is based on geometric data.

In at least one example, the tablet is administered to the subject in a fasted state.

In at least one example, the tablet is administered to the subject at least 30 minutes before and/or after eating food, such as at least 60 minutes before and/or after eating food.

In at least one example, the tablet is administered to the subject at least 2 hours before and/or after eating food.

In at least one example, the tablet is administered to the subject at least 2 hours before and after eating food.

In at least one example, the tablet is administered to the subject at least 10 hours after eating food and at least 4 hours before eating food.

In at least one example, the extended release pharmaceutical composition comprises:
about 30 to 75% w/w of the matrix polymer,
about 20 to 60% w/w of the permeability-modifying filler,
   wherein the permeability-modifying filler is slightly soluble, very slightly soluble, practically insoluble, or insoluble in water at about 20° C.

In at least one example, the extended release pharmaceutical composition comprises:
about 35 to 75% w/w of the matrix polymer,
about 25 to 60% w/w of the permeability-modifying filler,
   wherein the permeability-modifying filler is practically insoluble or insoluble in water at about 20° C.

In at least one example, the extended release pharmaceutical composition comprises:
about 35 to 70% w/w of the matrix polymer,
about 25 to 55% w/w of the permeability-modifying filler,
   wherein the permeability-modifying filler is practically insoluble or insoluble in water at about 20° C.

In at least one example, the extended release pharmaceutical composition comprises:
about 30 to 78% w/w of the matrix polymer,
the permeability-modifying filler,
   wherein the extended release pharmaceutical composition is a tablet with a mass of about 300 to 600 mg.

In at least one example, the extended release pharmaceutical composition comprises:
about 30 to 75% w/w of the matrix polymer, and
about 20 to 60% w/w of the permeability-modifying filler,
   wherein the permeability-modifying filler is slightly soluble, very slightly soluble, practically insoluble or insoluble in water at about 20° C., and wherein the extended release pharmaceutical composition is a tablet with a mass of about 300 to 600 mg.

In at least one example, the extended release pharmaceutical composition comprises:
about 35 to 75% w/w of the matrix polymer,
about 25 to 60% w/w of the permeability-modifying filler,
   wherein the permeability-modifying filler is practically insoluble or insoluble in water at about 20° C., and wherein the extended release pharmaceutical composition is a tablet with a mass of about 300 to 600 mg.

In at least one example, the extended release pharmaceutical composition comprises:
about 35 to 70% w/w of the matrix polymer,
about 25 to 55% w/w of the permeability-modifying filler,
   wherein the permeability-modifying filler is practically insoluble or insoluble in water at about 20° C., and wherein the extended release pharmaceutical composition is a tablet with a mass of about 300 to 600 mg.

In at least one example, the extended release pharmaceutical composition comprises:
about 30 to 75% w/w of the matrix polymer, and
about 20 to 60% w/w of the permeability-modifying filler,
   wherein the permeability-modifying filler is slightly soluble, very slightly soluble, practically insoluble or insoluble in water at about 20° C., wherein the extended release pharmaceutical composition is a tablet with a mass of about 300 to 600 mg, and/or comprises about 50 to 80 mg of ketamine.

In at least one example, the extended release pharmaceutical composition comprises:
about 35 to 75% w/w of the matrix polymer,
about 25 to 60% w/w of the permeability-modifying filler,
   wherein the permeability-modifying filler is practically insoluble or insoluble in water at about 20° C., wherein the extended release pharmaceutical composition is a tablet with a mass of about 300 to 600 mg, and/or comprises about 50 to 80 mg of ketamine.

In at least one example, the extended release pharmaceutical composition comprises:
about 35 to 70% w/w of the matrix polymer,
about 25 to 55% w/w of the permeability-modifying filler,
   wherein the permeability-modifying filler is practically insoluble or insoluble in water at about 20° C., wherein the extended release pharmaceutical composition is a tablet with a mass of about 300 to 600 mg, and/or comprises about 50 to 80 mg of ketamine.
   In at least one example, the extended release pharmaceutical composition comprises:
about 35 to 70% w/w of the matrix polymer,
about 25 to 55% w/w of the permeability-modifying filler,
wherein the matrix polymer is selected from one or more of the group hydroxypropyl methylcellulose, a carbomer homopolymer, and hydroxypropylcellulose,
   wherein the permeability-modifying filler is practically insoluble or insoluble in water at about 20° C.,
wherein the permeability-modifying filler is selected from one or more of microcrystalline cellulose, starch (for example, without limitation, corn starch, pea starch, potato starch, rice starch, tapioca starch, wheat starch, arrowroot starch), calcium carbonate, and dicalcium phosphate,
wherein the extended release pharmaceutical composition is a tablet with a mass of about 300 to 600 mg, and/or comprises about 50 to 80 mg of ketamine.
   In at least one example, the extended release pharmaceutical composition comprises:
about 35 to 70% w/w of the matrix polymer,
about 25 to 55% w/w of the permeability-modifying filler,
wherein the matrix polymer is selected from one or more of the group hydroxypropyl methylcellulose, a carbomer homopolymer, and hydroxypropylcellulose,
wherein the permeability-modifying filler is practically insoluble or insoluble in water at about 20° C.,
wherein the permeability-modifying filler is selected from one or more of microcrystalline cellulose, starch (for example, without limitation, corn starch, pea starch, potato starch, rice starch, tapioca starch, wheat starch, arrowroot starch), calcium carbonate, and dicalcium phosphate,
wherein the extended release pharmaceutical composition is a tablet with a mass of about 300 to 600 mg, and/or comprises about 50 to 80 mg of ketamine,
wherein the extended release pharmaceutical composition exhibits an in vitro release with at least one of the following:
(a) about 25 to 35% of the active substance is released at about the 1 hour time point,
(b) about 36 to 50% of the active substance is released at about the 2 hour time point,
(c) about 53 to 73% of the active substance is released at about the 4 hour time point,
(d) about 64 to 88% of the active substance is released at about the 6 hour time point,
(e) about 73 to 99% of the active substance is released at about the 8 hour time point,
(f) about 79 to 100% of the active substance is released at about the 10 hour time point,
(g) about 81 to 100% of the active substance is released at about the 12 hour time point,
   when tested according to the USP Apparatus 1 (basket) method at a rotation rate of 100 rpm in media at pH 1.0 at 37.0±0.5° C.

In at least one example, the extended release pharmaceutical composition exhibits an in vitro release with at least two of (a) to (g).

In at least one example, the extended release pharmaceutical composition exhibits an in vitro release with at least three of (a) to (g).

In at least one example, the extended release pharmaceutical composition comprises:
about 35 to 70% w/w of the matrix polymer,
about 25 to 55% w/w of the permeability-modifying filler,
wherein the matrix polymer is selected from one or more of the group hydroxypropyl methylcellulose, a carbomer homopolymer, and hydroxypropylcellulose,
wherein the permeability-modifying filler is practically insoluble or insoluble in water at about 20° C.,
wherein the permeability-modifying filler is selected from one or more of microcrystalline cellulose, starch (for example, without limitation, corn starch, pea starch, potato starch, rice starch, tapioca starch, wheat starch, arrowroot starch), calcium carbonate, and dicalcium phosphate,
wherein the extended release pharmaceutical composition is a tablet with a mass of about 300 to 600 mg, and/or comprises about 50 to 80 mg of ketamine,
wherein administration of the extended release pharmaceutical composition to a subject providing a dose of about 180 mg of ketamine results in any one or more of:
   a ketamine plasma concentration profile AUC of about 270 to 370 ng·hr/ml,
   a ketamine plasma concentration profile AUC_{0-∞} of about 270 to 370 ng·hr/ml, or about 280 to 360 ng·hr/ml,
   a ketamine plasma concentration profile AUC₀₋ₜ of about 270 to 370 ng·hr/ml, or about 270 to 355 ng·hr/ml,
   a norketamine plasma concentration profile AUC of about 3000 to 4000 ng·hr/ml,
   a norketamine plasma concentration profile AUC_{0-∞} of about 3000 to 4000 ng·hr/ml, or about 3500 to 3900 ng·hr/ml,
   a norketamine plasma concentration profile AUC₀₋ₜ of about 3000 to 4000 ng·hr/ml, or about 3300 to 3800 ng·hr/ml,
   a ketamine plasma concentration profile Cₘₐₓ of about 20 to 50 ng/ml, or about 25 to 40 ng/ml,
   a norketamine plasma concentration profile Cₘₐₓ of about 250 to 320 ng/ml, or about 260 to 300 ng/ml,
   a ketamine plasma concentration profile Tₘₐₓ of about 1.5 to 2.5 hours, or about 1.5 to 2.4 hours,
   a norketamine plasma concentration profile Tₘₐₓ of about 2 to 3 hours, or about 2 to 2.9 hours,
   a ketamine plasma concentration profile t_{1/2} of about 7 to 8 hours, or about 7.2 to 7.8 hours,
   a norketamine plasma concentration profile t_{1/2} of about 7.5 to 8.5 hours, or about 7.8 to 8.1 hours,
   a plasma concentration profile with a ratio of AUC norketamine/ketamine of about 8 to 15, or about 9 to 13 (for example a ratio of AUC ketamine to AUC norketamine of between about 1:8 and 1:15, or about 1:9 and 1:13),
   a plasma concentration profile with a ratio of AUC_{0-∞} norketamine/ketamine of about 8 to 15, or about 9 to 13 (for example a ratio of AUC_{0-∞} ketamine to AUC_{0-∞} norketamine of between about 1:8 and 1:15, or about 1:9 and 1:13),
   a plasma concentration profile with a ratio of AUC₀₋ₜ norketamine/ketamine of about 8 to 15, or about 9 to 13 (for example a ratio of AUC₀₋ₜ ketamine to AUC₀₋ₜ norketamine of between about 1:8 and 1:15, or about 1:9 and 1:13),
   a plasma concentration profile with a ratio of Cₘₐₓ norketamine/ketamine of about 6 to 10, or about 7 to 10 (for example a ratio of Cₘₐₓ ketamine to Cₘₐₓ norketamine of between about 1:6 and 1:10, or about 1:7 and 1:10).

In at least one example, the extended release pharmaceutical composition comprises:
about 35 to 70% w/w of the matrix polymer,
about 25 to 55% w/w of the permeability-modifying filler,
wherein the matrix polymer comprises hydroxypropyl methylcellulose (HPMC),
wherein the permeability-modifying filler is microcrystalline cellulose,
wherein the extended release pharmaceutical composition is a tablet with a mass of about 300 to 600 mg, and/or comprises about 50 to 80 mg of ketamine.

In at least one example, the extended release pharmaceutical composition comprises:
about 35 to 70% w/w of the matrix polymer,
about 25 to 55% w/w of the permeability-modifying filler,
wherein the matrix polymer comprises hydroxypropyl methylcellulose (HPMC),
wherein the permeability-modifying filler is microcrystalline cellulose,
wherein the extended release pharmaceutical composition is a tablet with a mass of about 300 to 600 mg, and/or comprises about 50 to 80 mg of ketamine,
wherein the extended release pharmaceutical composition exhibits an in vitro release with at least one of the following:
(a) about 25 to 35% of the active substance is released at about the 1 hour time point,
(b) about 36 to 50% of the active substance is released at about the 2 hour time point,
(c) about 53 to 73% of the active substance is released at about the 4 hour time point,
(d) about 64 to 88% of the active substance is released at about the 6 hour time point,
(e) about 73 to 99% of the active substance is released at about the 8 hour time point,
(f) about 79 to 100% of the active substance is released at about the 10 hour time point,
(g) about 81 to 100% of the active substance is released at about the 12 hour time point,
   when tested according to the USP Apparatus 1 (basket) method at a rotation rate of 100 rpm in media at pH 1.0 at 37.0±0.5° C.

In at least one example, the extended release pharmaceutical composition exhibits an in vitro release with at least two of (a) to (g).

In at least one example, the extended release pharmaceutical composition exhibits an in vitro release with at least three of (a) to (g).

In at least one example, the extended release pharmaceutical composition comprises:
about 35 to 70% w/w of the matrix polymer,
about 25 to 55% w/w of the permeability-modifying filler,
wherein the matrix polymer comprises hydroxypropyl methylcellulose (HPMC),
wherein the permeability-modifying filler is microcrystalline cellulose,
wherein the extended release pharmaceutical composition is a tablet with a mass of about 300 to 600 mg, and/or comprises about 50 to 80 mg of ketamine,
wherein administration of the extended release pharmaceutical composition to a subject providing a dose of about 180 mg of ketamine results in any one or more of:
   a ketamine plasma concentration profile AUC of about 270 to 370 ng·hr/ml,
   a ketamine plasma concentration profile AUC_{0-∞} of about 270 to 370 ng·hr/ml, or about 280 to 360 ng·hr/ml,
   a ketamine plasma concentration profile AUC₀₋ₜ of about 270 to 370 ng·hr/ml, or about 270 to 355 ng·hr/ml,
   a norketamine plasma concentration profile AUC of about 3000 to 4000 ng·hr/ml,
   a norketamine plasma concentration profile AUC_{0-∞} of about 3000 to 4000 ng·hr/ml, or about 3500 to 3900 ng·hr/ml,
   a norketamine plasma concentration profile AUC₀₋ₜ of about 3000 to 4000 ng·hr/ml, or about 3300 to 3800 ng·hr/ml,
   a ketamine plasma concentration profile Cₘₐₓ of about 20 to 50 ng/ml, or about 25 to 40 ng/ml,
   a norketamine plasma concentration profile Cₘₐₓ of about 250 to 320 ng/ml, or about 260 to 300 ng/ml,
   a ketamine plasma concentration profile Tₘₐₓ of about 1.5 to 2.5 hours, or about 1.5 to 2.4 hours,
   a norketamine plasma concentration profile Tₘₐₓ of about 2 to 3 hours, or about 2 to 2.9 hours,
   a ketamine plasma concentration profile t_{1/2} of about 7 to 8 hours, or about 7.2 to 7.8 hours,
   a norketamine plasma concentration profile t_{1/2} of about 7.5 to 8.5 hours, or about 7.8 to 8.1 hours,
   a plasma concentration profile with a ratio of AUC norketamine/ketamine of about 8 to 15, or about 9 to 13 (for example a ratio of AUC ketamine to AUC norketamine of between about 1:8 and 1:15, or about 1:9 and 1:13),
   a plasma concentration profile with a ratio of AUC_{0-∞} norketamine/ketamine of about 8 to 15, or about 9 to 13 (for example a ratio of AUC_{0-∞} ketamine to AUC_{0-∞} norketamine of between about 1:8 and 1:15, or about 1:9 and 1:13),
   a plasma concentration profile with a ratio of AUC₀₋ₜ norketamine/ketamine of about 8 to 15, or about 9 to 13 (for example a ratio of AUC₀₋ₜ ketamine to AUC₀₋ₜ norketamine of between about 1:8 and 1:15, or about 1:9 and 1:13),
   a plasma concentration profile with a ratio of Cₘₐₓ norketamine/ketamine of about 6 to 10, or about 7 to 10 (for example a ratio of Cₘₐₓ ketamine to Cₘₐₓ norketamine of between about 1:6 and 1:10, or about 1:7 and 1:10).

In at least one example, the extended release pharmaceutical composition comprises:
about 45 to 80% w/w of the matrix polymer, and
about 3 to 35% w/w of the permeability-modifying filler,
   wherein the permeability-modifying filler is very soluble or freely soluble in water at about 20° C.

In at least one example, the extended release pharmaceutical composition comprises:
about 50 to 80% w/w of the matrix polymer, and
about 3 to 30% w/w of the permeability-modifying filler,
   wherein the permeability-modifying filler is very soluble or freely soluble in water at about 20° C.

In at least one example, the extended release pharmaceutical composition comprises:
about 60 to 80% w/w of the matrix polymer, and
about 3 to 20% w/w of the permeability-modifying filler,
   wherein the permeability-modifying filler is very soluble or freely soluble in water at about 20° C.

In at least one example, the extended release pharmaceutical composition comprises:
about 70 to 80% w/w of the matrix polymer, and
about 3 to 15% w/w of the permeability-modifying filler,
   wherein the permeability-modifying filler is very soluble or freely soluble in water at about 20° C.

In at least one example, the extended release pharmaceutical composition comprises:
about 45 to 80% w/w of the matrix polymer, and
about 3 to 35% w/w of the permeability-modifying filler,
   wherein the permeability-modifying filler is very soluble or freely soluble in water at about 20° C., and
   wherein the extended release pharmaceutical composition is a tablet with a mass of about 300 to 600 mg.

In at least one example, the extended release pharmaceutical composition comprises:
about 50 to 80% w/w of the matrix polymer, and
about 3 to 30% w/w of the permeability-modifying filler,
   wherein the permeability-modifying filler is very soluble or freely soluble in water at about 20° C., and
   wherein the extended release pharmaceutical composition is a tablet with a mass of about 300 to 600 mg.

In at least one example, the extended release pharmaceutical composition comprises:
about 60 to 80% w/w of the matrix polymer, and
about 3 to 20% w/w of the permeability-modifying filler,
   wherein the permeability-modifying filler is very soluble or freely soluble in water at about 20° C., and
   wherein the extended release pharmaceutical composition is a tablet with a mass of about 300 to 600 mg.

In at least one example, the extended release pharmaceutical composition comprises:
about 70 to 80% w/w of the matrix polymer, and
about 3 to 15% w/w of the permeability-modifying filler,
   wherein the permeability-modifying filler is very soluble or freely soluble in water at about 20° C., and
   wherein the extended release pharmaceutical composition is a tablet with a mass of about 300 to 600 mg.

In at least one example, the extended release pharmaceutical composition comprises:
about 45 to 80% w/w of the matrix polymer, and
about 3 to 35% w/w of the permeability-modifying filler,
   wherein the permeability-modifying filler is very soluble or freely soluble in water at about 20° C., and wherein the extended release pharmaceutical composition is a tablet comprising about 50 to 80 mg of ketamine.

In at least one example, the extended release pharmaceutical composition comprises:
about 50 to 80% w/w of the matrix polymer, and
about 3 to 35% w/w of the permeability-modifying filler,
   wherein the permeability-modifying filler is very soluble or freely soluble in water at about 20° C., and wherein the extended release pharmaceutical composition is a tablet comprising about 50 to 80 mg of ketamine.

In at least one example, the extended release pharmaceutical composition comprises:
about 60 to 80% w/w of the matrix polymer, and
about 3 to 20% w/w of the permeability-modifying filler,
   wherein the permeability-modifying filler is very soluble or freely soluble in water at about 20° C. and wherein the extended release pharmaceutical composition is a tablet comprising about 50 to 80 mg of ketamine.

In at least one example, the extended release pharmaceutical composition comprises:
about 70 to 80% w/w of the matrix polymer, and
about 3 to 15% w/w of the permeability-modifying filler,
   wherein the permeability-modifying filler is very soluble or freely soluble in water at about 20° C., and wherein the extended release pharmaceutical composition is a tablet comprising about 50 to 80 mg of ketamine.

In at least one example, the extended release pharmaceutical composition comprises:
about 45 to 80% w/w of the matrix polymer, and
about 3 to 35% w/w of the permeability-modifying filler,
   wherein the permeability-modifying filler is very soluble or freely soluble in water at about 20° C., and
   wherein the extended release pharmaceutical composition is a tablet with a mass of about 300 to 600 mg, and/or comprises about 50 to 80 mg of ketamine.

In at least one example, the extended release pharmaceutical composition comprises:
about 50 to 80% w/w of the matrix polymer, and
about 3 to 30% w/w of the permeability-modifying filler,
   wherein the permeability-modifying filler is very soluble or freely soluble in water at about 20° C., wherein the extended release pharmaceutical composition is a tablet with a mass of about 300 to 600 mg, and/or comprises about 50 to 80 mg of ketamine.

In at least one example, the extended release pharmaceutical composition comprises:
about 60 to 80% w/w of the matrix polymer, and
about 3 to 20% w/w of the permeability-modifying filler,
   wherein the permeability-modifying filler is very soluble or freely soluble in water at about 20° C., wherein the extended release pharmaceutical composition is a tablet with a mass of about 300 to 600 mg, and/or comprises about 50 to 80 mg of ketamine.

In at least one example, the extended release pharmaceutical composition comprises:
about 70 to 80% w/w of the matrix polymer, and
about 3 to 15% w/w of the permeability-modifying filler,
   wherein the permeability-modifying filler is very soluble or freely soluble in water at about 20° C., wherein the extended release pharmaceutical composition is a tablet with a mass of about 300 to 600 mg, and/or comprises about 50 to 80 mg of ketamine.

In at least one example, the extended release pharmaceutical composition comprises:
the matrix polymer,
about 40 to 65% w/w of the permeability-modifying filler,
   wherein the extended release pharmaceutical composition is a tablet with a mass of about 700 to 1000 mg.

In at least one example, the extended release pharmaceutical composition comprises:
about 20 to 45% w/w matrix polymer,
about 40 to 65% w/w of the permeability-modifying filler,
   wherein the extended release pharmaceutical composition is a tablet with a mass of about 700 to 1000 mg.

In at least one example, the extended release pharmaceutical composition comprises:
about 25 to 40% w/w matrix polymer,
about 40 to 60% w/w of the permeability-modifying filler,
   wherein the extended release pharmaceutical composition is a tablet with a mass of about 700 to 1000 mg.

In at least one example, the extended release pharmaceutical composition comprises:
about 20 to 40% w/w matrix polymer,
about 40 to 60% w/w of the permeability-modifying filler,
   wherein the extended release pharmaceutical composition is a tablet with a mass of about 700 to 1000 mg.

In at least one example, the extended release pharmaceutical composition comprises:
about 20 to 45% w/w matrix polymer,
about 40 to 65% w/w of the permeability-modifying filler,
   wherein the extended release pharmaceutical composition is a tablet with a mass of about 700 to 1000 mg, and wherein the permeability-modifying filler is slightly soluble, very slightly soluble, practically insoluble, or insoluble in water at about 20° C.

In at least one example, the extended release pharmaceutical composition comprises:
about 25 to 40% w/w matrix polymer,
about 40 to 60% w/w of the permeability-modifying filler,
   wherein the extended release pharmaceutical composition is a tablet with a mass of about 700 to 1000 mg, and wherein the permeability-modifying filler is slightly soluble, very slightly soluble, practically insoluble, or insoluble in water at about 20° C.
   In at least one example, the extended release pharmaceutical composition comprises:
about 20 to 40% w/w matrix polymer,
about 40 to 60% w/w of the permeability-modifying filler,
   wherein the extended release pharmaceutical composition is a tablet with a mass of about 700 to 1000 mg, and wherein the permeability-modifying filler is slightly soluble, very slightly soluble, practically insoluble, or insoluble in water at about 20° C.

In at least one example, the extended release pharmaceutical composition comprises:
about 20 to 45% w/w matrix polymer,
about 40 to 65% w/w of the permeability-modifying filler,
   wherein the extended release pharmaceutical composition is a tablet with a mass of about 700 to 1000 mg and/or comprises about 100 to 140 mg of ketamine, and wherein the permeability-modifying filler is slightly soluble, very slightly soluble, practically insoluble, or insoluble in water at about 20° C.

In at least one example, the extended release pharmaceutical composition comprises:
about 25 to 40% w/w matrix polymer,
about 40 to 60% w/w of the permeability-modifying filler,
   wherein the extended release pharmaceutical composition is a tablet with a mass of about 700 to 1000 mg and/or comprises about 100 to 140 mg of ketamine, and wherein the permeability-modifying filler is slightly soluble, very slightly soluble, practically insoluble, or insoluble in water at about 20° C.

In at least one example, the extended release pharmaceutical composition comprises one or more of a lubricant, a granulating aid, and/or a glidant.

In at least one example, the lubricant is selected from one or more of magnesium stearate, calcium stearate, and zinc stearate.

In at least one example, the extended release pharmaceutical composition comprises about 0.1 to 5% w/w of one or more lubricant(s).

In at least one example, the glidant includes silicon dioxide.

In at least one example, the extended release pharmaceutical composition comprises about 0.01 to 5% w/w of one or more glidant(s).

In at least one example, the tablet additionally includes a coating.

In at least one example, the coating improves ease of swallowing.

In at least one example, the coating improves ease of processing and/packaging the tablet.

In at least one example, the coating comprises hydroxypropyl methylcellulose and polyethylene glycol. In at least one example, the coating comprises hydroxypropyl methylcellulose, titanium dioxide, and polyethylene glycol.

In at least one example, the coating is an additional about 1 to 8% w/w of the tablet. In at least one example, the coating is an additional about 2 to 8% w/w of the tablet. In at least one example, the coating is an additional about 3 to 5% w/w of the tablet.

In at least one example, the tablet with the coating has a mass of about 202 to 1080 mg. In at least one example, the tablet with the coating has a mass of about 303 to 918 mg. In at least one example, the tablet with the coating has a mass of about 303 to 648 mg. In at least one example, the tablet with the coating has a mass of about 303 to 540 mg. In at least one example, the tablet with the coating has a mass of about 707 to 1080 mg. In at least one example, the tablet with the coating has a mass of about 707 to 972 mg. In at least one example, the tablet with the coating has a mass of about 412 to 420 mg. In at least one example, the tablet with the coating has a mass of about 824 to 840 mg.

In at least one example, the extended release pharmaceutical composition comprises about 90 to 110% of starting amount of the active agent at 6 months of storage at 40° C. with 75% relative humidity.

In at least one example, the extended release pharmaceutical composition comprises about 90 to 110% of starting amount of the active agent at 12 months of storage at 25° C. with 60% relative humidity.

In at least one example, the extended release pharmaceutical composition comprises about 90 to 110% of starting amount of the active agent at 24 months of storage at 25° C. with 60% relative humidity.

In at least one example, the amount of the active agent changes by less than or equal to about 10% following storage for 6 months at 40° C. at 75% relative humidity.

In at least one example, the amount of the active agent changes by less than or equal to about 10% following storage for 12 months at 25° C. at 60% relative humidity.

In at least one example, the amount of the active agent changes by less than or equal to about 10% following storage for 24 months at 25° C. at 60% relative humidity.

In at least one example storage is in a closed container.

In at least one example, the method of manufacturing an extended release pharmaceutical composition comprises dry blending the therapeutically effective amount of the active agent, the matrix polymer and the permeability-modifying filler.

In at least one example, the blend of the active agent, the matrix polymer and the permeability-modifying filler are sieved.

In at least one example, the blend of the active agent, the matrix polymer and the permeability-modifying filler are sieved through a 600 to 1000 μm screen.

In at least one example, the method of manufacturing an extended release pharmaceutical composition comprises adding a lubricant

In at least one example, the lubricant is added to the blend of the active agent, the matrix polymer and the permeability-modifying filler.

In at least one example, the method of manufacturing an extended release pharmaceutical composition comprises forming the extended release pharmaceutical composition into a tablet.

In at least one example, the method of manufacturing an extended release pharmaceutical composition comprises directly compressing the blend of the active agent, the matrix polymer and the permeability-modifying filler into a tablet shape.

In at least one example, the method of manufacturing an extended release pharmaceutical composition comprises directly compressing the blend of the active agent, the matrix polymer, the permeability-modifying filler and the lubricant into a tablet shape.

In at least one example, the direct compression uses a compression force of about 10 to 50 kN. In at least one example, the direct compression uses a compression force of about 10 to 40 kN.

In at least one example, the method of manufacturing an extended release pharmaceutical composition comprises coating the pharmaceutical composition.

In at least one example, the method of manufacturing an extended release pharmaceutical composition comprises coating the tablet.

In at least one example, the method of manufacturing an extended release pharmaceutical composition comprises filling a capsule shell with the blend of the active agent, the matrix polymer and the permeability-modifying filler.

For the avoidance of doubt, the embodiments and/or examples may apply alone or in any combination of two or more thereof to any one or more of the aspects set forth above where the context allows.

Further aspects of the present invention and further embodiments and/or examples of the aspects described in the preceding paragraphs will become apparent from the following description, given by way of example and with reference to the accompanying drawings.

### BRIEF DESCRIPTION OF THE FIGURES

The invention will be described in conjunction with the following Figures:

FIG. 1 is a graph showing dissolution profiles of the comparative 60 mg PEO sustained release ketamine compositions at 3 different pHs.

FIG. 2 is a graph showing dissolution profiles of examples of compositions with different matrix polymers.

FIG. 3 is a graph showing dissolution profiles of examples of compositions with different permeability-modifying fillers.

FIG. 4 is a graph showing dissolution profiles of examples of compositions with different levels of HPMC, HPMC/HPC, and lactose.

FIG. 5 is a graph showing dissolution profiles of examples of compositions with different levels of mannitol as the permeability-modifying filler.

FIG. 6 is a graph showing dissolution profiles of examples of compositions with different levels of matrix polymer and different size tablets.

FIG. 7 is a graph showing mean ketamine and norketamine plasma concentration versus sample time.

### DEFINITION OF TERMS

As used herein the term “active pharmaceutical ingredient” (“API”) or “pharmaceutically active agent” or “active agent” or “active” is a drug or agent which can be employed as disclosed herein and is intended to be used in the human or animal body in order to heal, to alleviate, or to prevent diseases, ailments, or to influence the state, the condition or the functions of the body or mental states.

An amount is “effective” as used herein, when the amount provides an effect in the subject. As used herein, the term “effective amount” or “therapeutically effective” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan.

As used herein, “therapy” or “therapeutic” includes preventing, treating, and/or managing a condition in a subject. As used herein, the terms “therapies” and “therapy” can refer to any method(s), composition(s), and/or agent(s) that can be used in the prevention, treatment, and/or management of a disease or condition, or one or more symptoms thereof.

As used herein, the terms “prevent,” “preventing” and “prevention” in the context of the administration of a therapy to a subject refer to the prevention or inhibition of the recurrence or onset of a disease or condition, such as depression, anxiety, alcohol abuse disorder, and/or pain, or a symptom thereof in a subject resulting from the administration of a therapy (e.g., a prophylactic or therapeutic agent), or a combination of therapies (e.g., a combination of prophylactic or therapeutic agents).

As used herein, the terms “treat,” “treatment,” and “treating” in the context of the administration of a therapy to a subject refer to the reduction or inhibition of the progression and/or duration of a disease or condition, such as depression, anxiety, alcohol abuse disorder, and/or pain, and/or the amelioration of one or more symptoms thereof resulting from the administration of one or more therapies.

As used herein, the terms “manage”, “management” and “managing” in the context of the administration of a therapy to a subject refer to refer to the prevention or inhibition of the development of a disease or condition, such as depression, anxiety, alcohol abuse disorder, and/or pain, or a symptom thereof in a subject resulting from the administration of a therapy (e.g., a prophylactic or therapeutic agent), or a combination of therapies (e.g., a combination of prophylactic or therapeutic agents).

As used herein, the terms “subject” and “patient” are used interchangeably. As used herein, the term “patient” refers to an animal, preferably a mammal such as a non-primate (e.g., cows, pigs, horses, cats, dogs, rats etc.), and a primate (e.g., monkey and human), and most preferably a human. In at least one example, the subject is a non-human animal such as a farm animal (e.g., a horse, pig, or cow) or a pet (e.g., a dog or cat). In at least one example, the subject is an elderly human. In at least one example, the subject is a human adult. In at least one example, the subject is a human child. In at least one example, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “about” when used in conjunction with a stated numerical value or range has the meaning reasonably ascribed to it by a person skilled in the art, i.e. denoting somewhat more or somewhat less than the stated value or range, for example it may vary by as much as 15% or 10% or as much as 9%, 8%, 7%, 6%, 5%, 4%, 3%, 2%, 1%, to the stated numerical value or range.

It should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used herein, except where the context requires otherwise, the term “comprise” and variations of the term, such as “comprising”, “comprises” and “comprised”, are not intended to exclude further additives, components, integers or steps.

### DETAILED DESCRIPTION

Described herein is an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler.

Further or alternatively, described herein is an extended release pharmaceutical composition for prevention, treatment and/or management, of a subject in need thereof comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler.

Further or alternatively, described herein is a method of preventing, treating and/or managing depression, anxiety, alcohol abuse disorder, and/or pain in a subject, the method comprising administering an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler.

Further or alternatively, described herein is a method of preventing, treating and/or managing depression in a subject, the method comprising administering an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler.

Further or alternatively, described herein is a method of preventing, treating and/or managing treatment-resistant depression in a subject, the method comprising administering an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler.

Further or alternatively, described herein is use of an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, in the manufacture of a medicament for prevention, treatment and/or management of depression, anxiety, alcohol abuse disorder and/or pain.

Further or alternatively, described herein is use of an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, in the manufacture of a medicament for prevention, treatment, and/or management of depression.

Further or alternatively, described herein is use of an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, in the manufacture of a medicament for prevention, treatment, and/or management of treatment-resistant depression.

Further or alternatively, described herein is an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, for prevention, treatment and/or management of depression, anxiety, alcohol abuse disorder, and/or pain.

Further or alternatively, described herein is an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, for prevention, treatment, and/or management of depression.

Further or alternatively, described herein is an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, for prevention, treatment, and/or management of treatment-resistant depression.

Further or alternatively, described herein is use of an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, for prevention, treatment, and/or management of depression, anxiety, alcohol abuse disorder, and/or pain.

Further or alternatively, described herein is use of an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, for prevention, treatment, and/or management of depression.

Further or alternatively, described herein is use of an extended release pharmaceutical composition comprising a therapeutically effective amount of an active agent selected from the group consisting of ketamine, norketamine, pharmaceutically acceptable salts thereof, and combinations thereof, wherein the active agent is incorporated into a sustained-release polymeric matrix, the matrix comprising a matrix polymer, and a permeability-modifying filler, for prevention, treatment, and/or management of treatment-resistant depression.

Further or alternatively, described herein is a method of manufacturing an extended release pharmaceutical composition, the method comprising blending a therapeutically effective amount of an active agent selected from the group consisting of ketamine, norketamine, pharmaceutically acceptable salts thereof, and combinations thereof with a matrix polymer and a permeability-modifying filler.

As noted in the Background section of this specification, it would be beneficial to have a composition that provides the same or substantially equivalent pharmacokinetics as described in the BEDROC study using R-107 sponsored by Douglas Pharmaceuticals Limited that was published in Nature Medicine (Glue et al. “Extended-release ketamine tablets for treatment-resistant depression: a randomized placebo-controlled phase 2 trial”, Nature Medicine, Volume 30, July 2024, 2004-2009), without using a composition based on PEO.

### Active Agent

The pharmaceutical composition of the invention comprises an active agent, selected from the group consisting of ketamine, norketamine, pharmaceutically acceptable salts thereof, and combinations thereof.

“Ketamine” as used herein is understood to comprise the compound of Formula (I)

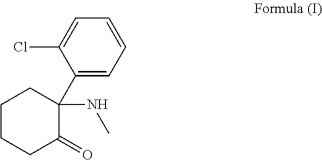 [SMILES: CNC1(c2ccccc2Cl)CCCCC1=O]

having the IUPAC name 2-(2-chlorophenyl)-2-(methylamino)cyclohexan-1-one. Accordingly, ketamine comprises the R and S enantiomers as well as pharmaceutically acceptable salts and/or solvates thereof.

The active agent may consist of (R)-ketamine and/or pharmaceutically acceptable salts and/or solvates thereof. The active agent may consist of (S)-ketamine and/or pharmaceutically acceptable salts and/or solvates thereof. The active agent may consist of a mixture of (S)-ketamine and (R)-ketamine and/or pharmaceutically acceptable salts and/or solvates thereof. The active agent may consist of a racemic mixture of (S)-ketamine and (R)-ketamine and/or pharmaceutically acceptable salts and/or solvates thereof.

Norketamine has the IUPAC name 2-amino-2-(2-chlorophenyl)cyclohexan-1-one of formula (II)

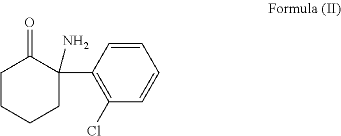 [SMILES: NC1(c2ccccc2Cl)CCCCC1=O]

and is obtained from ketamine through N-demethylation. Norketamine may be (R)-norketamine and/or pharmaceutically acceptable salts and/or solvates thereof, or (S)-norketamine and/or pharmaceutically acceptable salts and/or solvates thereof, racemate of (S)-norketamine and (R)-norketamine and/or pharmaceutically acceptable salts and/or solvates thereof, or a mixture of (S)-norketamine and (R)-norketamine and/or pharmaceutically acceptable salts and/or solvates thereof.

The pharmaceutically acceptable salts of ketamine and/or norketamine may comprise the pharmaceutically acceptable acid addition salts. The acids which are used to prepare the pharmaceutically acceptable acid addition salts may be those which form non-toxic acid addition salts, i.e. salts containing pharmacologically acceptable anions, such as chloride, bromide, iodide, nitrate, sulfate, bisulfate, phosphate, acid phosphate, acetate, lactate, citrate, (D,L)- and L-tartrate, (D,L)- and L-malate, bitartrate, succinate, maleate, fumarate, gluconate, saccharate and benzoate. A preferred salt is the hydrochloride of ketamine.

Reference to the percentage or amount of “active agent”, “ketamine” or “norketamine”, in the composition, without specifying a salt, should be taken to be the percentage or amount of free active agent, ketamine or norketamine. For example, a composition comprising about 17.3% w/w ketamine HCl contains about 15% w/w free ketamine.

Within the patient, after consuming the tablet, ketamine and/or norketamine may form or break down into various metabolites. Once within the patient, “ketamine” as used herein may also comprise its metabolites. The metabolite may be norketamine, hydroxnorketamine, dehydroxyketamine and/or dehydronorketamine, preferably norketamine. Testing of the pharmacokinetics of ketamine and/or norketamine compositions may therefore include measurement and/or testing of metabolites.

Ketamine is generally a water soluble active (for example, ketamine hydrochloride has a solubility in water of 20 g/100 mL). Without addition into an extended release composition, soluble ketamine will be naturally prone to fast release. The inventors found the extended-release composition described herein required combining of several variables to provide the same or substantially equivalent pharmacokinetics as R-107. Variation of one variable led to a cascade effect requiring unexpected change to other variables.

### Matrix Polymer

The sustained-release polymeric matrix may be described as an erodible polymeric matrix. “Erodible matrix” should be taken to mean an aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible matrix imbibes water and forms an aqueous-swollen gel or matrix that entraps the active agent. The aqueous-swollen matrix gradually erodes, swells, disintegrates, dissolves in the environment of use, thereby controlling the release of the active agent to the environment of use. Active agent release may occur by a variety of mechanisms: the matrix may disintegrate or dissolve from around particles or granules of the active; or the active may dissolve in the aqueous solution and/or gel and diffuse from the tablet.

A key ingredient of this water-swollen matrix is the water-swellable, erodible, or soluble, matrix polymer, which may generally be described as an osmopolymer, hydrophilic, hydrogel, or water-swellable polymer.

The sustained-release polymeric matrix is made up of least the active agent, a matrix polymer, and a permeability-modifying filler (but may include other components) but is preferably blended/mixed to be substantially homogeneous.

The matrix polymer (and/or mixture of matrix polymers) may have a viscosity of equal to or greater than about 2,000 mPa·s, or equal to or greater than about 3,000 mPa·s, or equal to or greater than about 4,000 mPa·s (when measured at about 1% w/v in water at about 20° C.). The matrix polymer (and/or mixture of matrix polymers) may have viscosity of about 2,000 to 150,000, about 3,000 to 150,000, about 4,000 to 150,000 mPa·s (when measured at about 1% w/v in water at about 20° C.).

The matrix polymer may comprise a hypromellose (hydroxypropyl methylcellulose, HPMC), a carbomer homopolymer Type B, a carbomer homopolymer Type C, and/or hydroxypropyl cellulose.

Examples of matrix polymers are shown in Table 1.

The hypromellose is defined in USP 40-NF 35 S1. The hypromellose used in the composition may have a viscosity of equal to or greater than about 4,000 mPa·s, or equal to or greater than about 8,000 mPa·s, or equal to or greater than about 10,000 mPa·s, equal to or greater than about than about 50,000 mPa·s (when measured at about 2% w/v in water at about 20° C.). The hypromellose may have a viscosity of about 4,000 to 300,000 mPa·s, about 6,000 to 300,000 mPa·s, about 8,000 to 300,000 mPa·s, about 10,000 to 300,000 mPa·s, or about 50,000 to 200,000 mPa·s (when measured at about 2% w/v in water at about 20° C.). The conditions for measurement of the viscosity of hypromellose (HPMC, hypromellose) are defined in USP 40-NF 35 Si (Method 2—rotational method—for samples with viscosity over 600 mPa·s, measurement in about 2% w/v water at about 20° C.).

Carbomer homopolymer are categorized into Type A, B and C partly based on their viscosity characteristics, with carbomer homopolymers A having a viscosity of 4,000-11,000 mPa·s, B a viscosity of 29,400-39,400 mPa·s and C a viscosity of 40,000-60,000 mPa·s (2.50 g carbomer in 500 mL (about 0.5% w/v) of water at about 25° C.) measured according to the conditions defined in USP 40-NF 35 Si.

The carbomer homopolymer Type B or C used in the composition may have a viscosity of equal to or greater than about 29,400 mPa·s, or about 29,400 to 60,000 mPa·s, (when measured at about 2% w/v in water at about 20° C.). Viscosity may be measured according to the conditions defined in USP 40-NF 35 Si.

The hydroxypropyl cellulose may have a viscosity of equal to or greater than about 2,000, or equal to or greater than about 4,000 mPa·s (when measured at 1% w/v in water at 25° C.). If an hydroxypropyl cellulose is used with a lower viscosity it is preferably combined with another matrix polymer with a higher viscosity (for example, without limitation, a hydroxypropyl methylcellulose, carbomer homopolymer Type B or C), such that the viscosity of the matrix polymers may be equal to or greater than about 2,000, or equal to or greater than about 4,000 mPa·s (when measured at about 1% w/v in water at about 25° C.).

TABLE 1
Examples of matrix polymers

| Matrix polymer | Description | Grade | Viscosity (mPa · s) |
|---|---|---|---|
| Hydroxypropyl | HPMC, | Methocel Dow Wolff | 75,000-140,000 |
| methylcellulose | Hypromellose, | Cellulosics | (2% w/v in water at |
| | Methocel ™ | K100M | 20° C.) |
| | Benecel ™ | Methocel Dow Wolff | 10,000-18,000 |
| | | Cellulosics | (2% w/v in water at |
| | | K15M | 20° C.) |
| | | Ashland | 150,000-280,000 |
| | | Benecel ™ | (2% w/v in water at |
| | | K200M | 20° C.) |
| | | Ashland | 26,250-49,000 |
| | | Benecel ™ | (2% w/v in water at |
| | | K35M | 20° C.) |
| | | Methocel Dow Wolff | 4,000 (2% w/v in |
| | | Cellulosics K4M | water at 20° C.) |
| Carbomer | Carbopol ®, | Type B | 29,400-39,400 (0.5% |
| Homopolymer | Carboxyvinyl | | w/v at 25° C.) |
| Type B | Polymer, | 974P NF | 29,400-39,400 (0.5 |
| | Allyl Pentaerythritol | 974 NF | w/v % at pH 7.5) |
| | Crosslinked | | |
| Carbomer | Carboxyvinyl | Type C | 40,000-60,000 (0.5% |
| Homopolymer | Polymer, | | w/v at 25° C.) |
| Type C | Allyl Pentaerythritol | Carbopol ® 980 NF | 40,000-60,000 (0.5% |
| | Crosslinked | | w/v at 25° C.) |
| Hydroxypropyl | HPC, Klucel ™ | HF, HXF (Klucel, | 1,500-3,000 |
| cellulose | | Ashland Specialty | (1% w/v in water at |
| | | Ingredients), | 25° C.*) |
| | | MW 1,150,000 | |
| | | EF (Klucel, Ashland | 300-600 mPa · s (10% |
| | | Specialty Ingredients), | in water at 25° C.) |
| | | MW 80,000 | |

*From Handbook of Pharmaceuticals Excipients, Eighth Edition, Edited by Paul J Sheskey, Walter G Cook and Colin G Cabel, Published Pharmaceutical Press and the American Pharmacists Association

### Permeability-Modifying Filler

The permeability-modifying filler is mixed with the matrix polymer to give a substantially homogeneous sustained-release polymeric matrix. The permeability-modifying filler generally increases the rate of dissolution of the active agent from the sustained-release polymeric matrix over matrix polymer alone. While not wishing to be bound by theory, it is believed the permeability-modifying filler disrupts the matrix, and allows faster erosion, disintegration, and/or disrupts the polymer diffusion layer formed during tablet dissolution.

The permeability-modifying filler may be selected from one or more of microcrystalline cellulose, starch (for example, without limitation, corn starch, pea starch, potato starch, rice starch, tapioca starch, wheat starch, arrowroot starch, etc.), pregelatinized starch, mannitol, polyvinylpyrrolidone, lactose (for example, without limitation, lactose monohydrate), fructose, dextrose (for example, without limitation, anhydrous dextrose, dextrose monohydrate), sorbitol, sucrose, maltodextrin, and calcium salts (for example, without limitation, calcium carbonate, calcium phosphate-dibasic, calcium phosphate-tribasic, calcium sulfate).

The polyvinylpyrrolidone may be a grade and/or molecular weight which is very soluble or freely soluble water, for example, without limitation, K12 or K15. The molecular weight of the polyvinylpyrrolidone may be about 5,000 to 10,000.

The permeability-modifying filler may be divided into generally water soluble (very soluble, or freely soluble) and generally water insoluble (insoluble, practically insoluble, very slightly soluble, or slightly soluble) when measured in water at 20° C. Definitions of the descriptive terms for solubility are provided in the USP-NF/PF and as shown in Table 2.

TABLE 2
Definitions of descriptive terms for solubility

| | Parts of solvent | Solubility |
|---|---|---|
| Descriptive term of | required for1 part | Range |
| solubility | of solute (by mass) | (mg/mL) |
| Very soluble | Less than 1 | >1000 |
| Freely soluble | From 1 to 10 | 100-1000 |
| Soluble | From 10 to 30 | 33-100 |
| Sparingly soluble | From 30 to 100 | 10-33 |
| Slightly soluble | From 100 to 1000 | 1-10 |
| Very slightly soluble | From 1000 to 10,000 | 0.1-1 |
| Practically insoluble, or | From 10,000 and over | <0.1 |
| insoluble | | |

Examples of permeability-modifying fillers are shown in Table 3.

TABLE 3
Examples of permeability-modifying fillers

| Permeability- | | Solubility in Water (at 20° C. unless |
|---|---|---|
| modifying filler | Description | otherwise stated) |
| microcrystalline | MCC102, Microcel ™ | practically insoluble in water* |
| cellulose | | |
| starch | corn starch, pea starch, potato | practically insoluble in cold water* |
| | starch, rice starch, tapioca | |
| | starch, wheat starch, | |
| | arrowroot starch | |
| calcium salts | calcium carbonate | practically insoluble in water* |
| | calcium phosphate-dibasic | practically insoluble in water* |
| | Dicalcium phosphate (DCP) | |
| | calcium phosphate-tribasic | Very slightly soluble in water* |
| | calcium sulfate (dihydrate) | 1 in 375 at 20° C.* (slightly soluble) |
| pregelatinized | | Slightly soluble in cold water, depending |
| starch | | upon degree of pregelatinization* |
| lactose | lactose hydrate, lactose | 1 in 5.24 at 20° C.* (freely soluble) |
| | monohydrate | |
| mannitol | | 1 in 5.5 at 20° C.* (freely soluble) |
| fructose | | 1 in 0.3 at 20° C.* (very soluble) |
| dextrose | D-glucose | 1 in 1 at 20° C.* (freely soluble) |
| sorbitol | | 1 in 0.5 at 20° C.* (very soluble) |
| sucrose | | 1 in 0.5 at 20° C.* (very soluble) |
| maltodextrin | | Freely soluble* |
| povidone | PVP, polyvinylpyrrolidone, | Freely soluble in water. In water, the |
| | polyvidone, Kollidon, | concentration of a solution is limited only |
| | Grades: K12 (MW about | by the viscosity of the resulting solution, |
| | 5,000) or K15 (MW about | which is a function of the K value.* |
| | 10,000) | |

*From Handbook of Pharmaceuticals Excipients, Eighth Edition, Edited by Paul J Sheskey, Walter G Cook and Colin G Cabel, Published Pharmaceutical Press and the American Pharmacists Association

The extended release composition may comprise about 3 to 60% w/w of the permeability-modifying filler.

As shown in Examples 3 and 4, to achieve a good match to the previous PEO composition, generally a higher amount of permeability-modifying filler which is slightly soluble, very slightly soluble, practically insoluble, or insoluble in water is desirable (and corresponding lower amount of matrix polymer). The extended release composition may comprise about 20 to 60% w/w of the permeability-modifying filler, wherein the permeability-modifying filler is slightly soluble, very slightly soluble, practically insoluble, or insoluble in water at about 20° C. The extended release composition may comprise about 30 to 60% w/w, or 30 to 55% w/w of the permeability-modifying filler, wherein the permeability-modifying filler is practically insoluble or insoluble in water at about 20° C. The permeability-modifying filler that is slightly soluble, very slightly soluble, practically insoluble, or insoluble in water at about 20° C. may be selected from one or more of microcrystalline cellulose, starch (for example, without limitation, corn starch, pea starch, potato starch, rice starch, tapioca starch, wheat starch, arrowroot starch), calcium salts (for example, without limitation, calcium carbonate, calcium phosphate-dibasic, calcium phosphate-tribasic, calcium sulfate), and pregelatinized starch.

An extended release composition made with a practically insoluble permeability-modifying filler (e.g. MCC) has shown good stability and ease of manufacturing (see Example 7).

As shown in Example 4, to achieve a good match to the previous PEO composition, generally a lower amount of permeability-modifying filler which is very soluble or freely soluble in water is desirable (and corresponding higher amount of matrix polymer). The extended release composition may comprise about 3 to 35% w/w, or about 3 to 20% w/w, or about 3 to 15% w/w of the permeability-modifying filler, wherein the permeability-modifying filler is very soluble or freely soluble in water at about 20° C. The permeability-modifying filler that is very soluble or freely soluble in water at about 20° C. is selected from one or more of lactose (for example, without limitation, lactose monohydrate), fructose, dextrose, sorbitol, sucrose, maltodextrin, mannitol, and polyvinylpyrrolidone. The permeability-modifying filler may be a sugar and/or a sugar alcohol that is solid at room temperature and pressure, and is very soluble or freely soluble in water at about 20° C., for example, without limitation, lactose, fructose, dextrose, sorbitol, sucrose, maltodextrin, and/or mannitol.

An extended release composition made with a freely soluble or vey soluble permeability-modifying filler, may provide the advantage of fewer insoluble particles in the composition if the composition is abused by crushing, dissolving, and injecting.

The extended release pharmaceutical composition may exhibit an in vitro release of one or more of (a) to (g):
(a) about 25 to 35% of the active substance is released at about the 1 hour time point,
(b) about 36 to 50% of the active substance is released at about the 2 hour time point,
(c) about 53 to 73% of the active substance is released at about the 4 hour time point,
(d) about 64 to 88% of the active substance is released at about the 6 hour time point,
(e) about 73 to 99% of the active substance is released at about the 8 hour time point,
(f) about 79 to 100% of the active substance is released at about the 10 hour time point,
(g) about 81 to 100% of the active substance is released at about the 12 hour time point,
   when tested according to the USP Apparatus 1 (basket) method at a rotation rate of 100 rpm in media at pH 1.0 at 37.0±0.5° C.

The extended release pharmaceutical composition may exhibit an in vitro release of:
about 41 to 50% of the active substance is released at about the 2 hour time point,
about 73 to 99% of the active substance is released at about the 8 hour time point, and
about 81 to 100% of the active substance is released at about the 12 hour time point,
   when tested according to the USP Apparatus 1 (basket) method at a rotation rate of 100 rpm in media at pH 1.0 at 37.0±0.5° C.

The extended release pharmaceutical composition may exhibit an in vitro release of one or more of (a) to (d):
(a) about 21 to 30 mg of ketamine at 2 hours,
(b) about 31 to 44 mg of ketamine at 4 hours,
(c) about 38 to 52 mg of ketamine at 6 hours,
(d) about 48 to 60 mg of ketamine at 12 hours,
   when tested according to the USP Apparatus 1 (basket) method at a rotation rate of 100 rpm in media at pH 1.0 at 37.0±0.5° C., wherein the extended release pharmaceutical composition is a tablet comprising about 60 mg of ketamine.

The extended release pharmaceutical composition may exhibit an in vitro release of one or more of (a) to (d):
(a) about 40 to 55 mg of ketamine at 2 hours,
(b) about 59 to 80 mg of ketamine at 4 hours,
(c) about 72 to 98 mg of ketamine at 6 hours,
(d) about 92 to 120 mg of ketamine at 12 hours,
   when tested according to the USP Apparatus 1 (basket) method at a rotation rate of 100 rpm in media at pH 1.0 at 37.0±0.5° C.,
wherein the extended release pharmaceutical composition is a tablet comprising about 120 mg of ketamine.

### Other Ingredients

In addition to the above ingredients, the pharmaceutical extended release composition may also contain suitable quantities of other materials, e.g., lubricants, granulating aids, and glidants.

Examples of lubricants include magnesium stearate, calcium stearate, and/or zinc stearate. The lubricant may be magnesium stearate.

Where a lubricant is included in the pharmaceutical composition, the amount of the lubricant may range from about 0.1 to 5% w/w of the pharmaceutical composition. The amount of the lubricant may range from about 0.1% to 3% w/w, about 0.1% to 2.5% w/w, about 0.1 to 2% w/w of the pharmaceutical composition. The amount of the lubricant may be about 0.1% w/w, about 0.2% w/w, about 0.3% w/w, about 0.4% w/w, about 0.5% w/w, about 1% w/w, about 1.5% w/w, about 2% w/w, about 2.5% w/w, about 3% w/w, about 3.5% w/w, about 4% w/w, about 4.5% w/w, or about 5% w/w of the extended release pharmaceutical composition.

An example of a glidant is colloidal silicon dioxide (e.g. Aerosil® silica by Evonik). Where a glidant is included in the pharmaceutical composition, the amount of the glidant may range from about 0.01 to 5% w/w of the extended release pharmaceutical composition.

### Tablet

The composition may be a tablet, for example, an oral tablet (for example a solid oral tablet). Each tablet may comprise about 30 mg, 60 mg, 120 mg, 180 mg or 240 mg of free ketamine.

Each tablet may have a mass of about 200 to 1000 mg, or 300 to 850 mg. For example, a tablet comprising about 60 mg of ketamine may have a mass of about 300 to 600 mg, about 300 to 500 mg, or about 400 mg. For example, a tablet comprising about 120 mg of ketamine may have a mass of about 700 to 1000 mg, about 700 to 900 mg, or about 800 mg.

The size of the tablet may affect the release of the active agent from the sustained-release polymeric matrix. While not wishing to be bound by theory, this may be due to the ratio of the surface area of the tablet exposed to fluids. Where the size of the tablet is increased the amount of matrix polymer may be decreased to increase the rate of release of the active agent. For example, the tablet may have a mass of about 300 to 600 mg (and/or about 30 to 60 mg of free ketamine) and comprises about 30 to 75% w/w, about 35 to 75% w/w, about 35 to 70% w/w, about 30 to 70% w/w of the matrix polymer. The tablet may have a mass of about 700 to 1000 mg (and/or about 120 to 180 mg of free ketamine) and comprises about 20 to 45% w/w, about 25 to 45% w/w, or about 25 to 40% w/w of the matrix polymer.

The tablet may have a crush strength of about 100 to 250 Newtons, or about 100 to 200 Newtons, or about 120 to 250 Newtons.

The tablet may be homogeneous, for example a substantially consistent matrix throughout. The tablet preferably does not have layers (except for a coating, see below), for example layers and/or core with different release profiles, or multitude of particles/pellets contained in a shell.

### Coating

The tablet may additionally comprise a coating. The coating may be a film coating, a sealant coating, a barrier coating, an enteric coating, a compress coating, a fast disintegrating coating, and/or an enzyme degradable coating. Multiple coatings may be applied for desired performance. Preferably, the coating does not have a significant effect on the release and/or pharmacokinetic profile of the tablet, for example comparison of dissolution profile with or without coating have an f₁ difference factor between about 0 and 15 and/or an f₂ similarity factor between about 50 and 100, for example when tested according to the USP Apparatus 1 (basket) method at a rotation rate of 100 rpm in media at pH 1.0 at 37.0±0.5° C.).

The coating may comprise a polymer, a plasticizer, a pigment or a combination thereof.

The coating may comprise polyvinyl alcohol (PVA), cellulose acetate phthalate (CAP), polyvinyl acetate phthalate (PVAP), methacrylic acid copolymers, cellulose acetate trimellitate (CAT), hydroxypropyl methylcellulose phthalate (HPMCP), hydroxypropyl methylcellulose (HPMC), hydroxypropyl methylcellulose acetate succinate, polyethylene glycol, shellac, sodium alginate, or zein. The coating may comprise hydroxypropyl methylcellulose and polyethylene glycol and optionally titanium dioxide. For example, the coating may comprise BPSI's Opadry® White Y-1-7000. For example, Opadry® Complete film coating system Y-1-700 White contains 62.5% w/w HPMC 2910/Hypromellose (E464, CFR ref. 172.874), 31.250% w/w Titanium dioxide (E171, CFR ref. 73.575, 73.1575, 73.2575), 6.250% w/w Macrogol/PEG (MW 400, E1521, CFR ref. 172.820).

The coating may be an additional mass of about 1 to 8% w/w of the tablet, or about 2 to 8% w/w of the tablet, or about 3 to 5% w/w of the tablet. The coating may be an additional mass of about 1, 2, 3, 4, 5, 6, 7, or 8% w/w of the tablet.

As discussed herein, reference to mass or amount of components of the extended release pharmaceutical composition should be taken to be mass or amount not including the coating unless specially stated otherwise. For example, where the tablet comprises 15% w/w free ketamine (which may be 17.3% w/w ketamine HCl), if the tablet had a coating of about 4% (range of about 3 to 5%) the amount of free ketamine in the coated tablet would be about 14.3 to 14.7% w/w.

As noted previously, the inventors found the extended-release composition described herein required combining and experimentation of several variables to provide the same or substantially equivalent pharmacokinetics as R-107. Various matrix polymers and various amounts/ratios of matrix polymer and permeability-modifying filler were tried. Variation of one variable led to a cascade effect requiring unexpected change to other variables. For example, if a water insoluble permeability-modifying filler was used (e.g. slightly soluble, very slightly soluble, practically insoluble, or insoluble in water at about 20° C.) a greater amount was needed than if a water soluble permeability-modifying filler was used (e.g. very soluble or freely soluble in water at about 20° C.). If the size and/or dose of the tablet was scaled up, the percentages of in vitro release changed, so the amount of matrix polymer was decreased.

In an example embodiment, the extended release pharmaceutical composition comprises:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
about 30 to 75% w/w of a matrix polymer,
about 20 to 60% w/w of a permeability-modifying filler,
   wherein the permeability-modifying filler is slightly soluble, very slightly soluble, practically insoluble, or insoluble in water at about 20° C.

In an example embodiment, the extended release pharmaceutical composition comprises:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
about 30 to 75% w/w of a matrix polymer,
about 20 to 60% w/w of a permeability-modifying filler,
   wherein the permeability-modifying filler is slightly soluble, very slightly soluble, practically insoluble, or insoluble in water at about 20° C., and wherein the pharmaceutical composition is a tablet with a mass of about 300 to 600 mg and/or the tablet comprises about 50 to 80 mg of ketamine.

In an example embodiment, the extended release pharmaceutical composition comprises:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
about 50 to 80% w/w of a matrix polymer, and
about 3 to 30% w/w of a permeability-modifying filler,
   wherein the permeability-modifying filler is very soluble or freely soluble in water at about 20° C.

In an example embodiment, the extended release pharmaceutical composition comprises:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
about 50 to 80% w/w of a matrix polymer, and
about 3 to 30% w/w of a permeability-modifying filler,
   wherein the permeability-modifying filler is very soluble or freely soluble in water at about 20° C. and
   wherein the pharmaceutical composition is a tablet with a mass of about 300 to 600 mg and/or comprises about 50 to 80 mg of ketamine.

In an example embodiment, the extended release pharmaceutical composition comprises:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
about 20 to 45% w/w of a matrix polymer, and
about 40 to 65% w/w of a permeability-modifying filler,
   wherein the pharmaceutical composition is a tablet with a mass of about 700 to 1000 mg and/or comprises about 100 to 140 mg of ketamine, and wherein the permeability-modifying filler is slightly soluble, very slightly soluble, practically insoluble, or insoluble in water at about 20° C.

### Pharmacokinetics

As previously noted, it would be beneficial to have a composition that provides the same or substantially equivalent pharmacokinetics as described in the BEDROC study using R-107 sponsored by Douglas Pharmaceuticals Limited that was published in Nature Medicine (Glue et al. “Extended-release ketamine tablets for treatment-resistant depression: a randomized placebo-controlled phase 2 trial”, Nature Medicine, Volume 30, July 2024, 2004-2009), without using a composition based on PEO.

The inventors were surprisingly able to match the pharmacokinetics of the composition based on PEO very closely, to be considered bioequivalent. For example, with 90% confidence intervals for AUC_{0-∞}, AUC₀₋ₜ, and/or Cₘₐₓ for ketamine in plasma and/or AUC_{0-∞}, AUC₀₋ₜ, and/or Cₘₐₓ for norketamine in plasma resulting from the comparison of the invention and the composition based on PEO being within the accepted bioequivalence criteria limits of 80.00% and 125.00% for log₁₀ transformed data.

Administration of the extended release pharmaceutical composition to a subject providing a dose of 180 mg of ketamine may result in any one or more of:
a ketamine plasma concentration profile AUC of about 270 to 370 ng·hr/ml,
a ketamine plasma concentration profile AUC_{0-∞} of about 270 to 370 ng·hr/ml, or about 280 to 360 ng·hr/ml,
a ketamine plasma concentration profile AUC₀₋ₜ of about 270 to 370 ng·hr/ml, or about 270 to 355 ng·hr/ml,
a norketamine plasma concentration profile AUC of about 3000 to 4000 ng·hr/ml,
a norketamine plasma concentration profile AUC_{0-∞} of about 3000 to 4000 ng·hr/ml, or about 3500 to 3900 ng·hr/ml,
a norketamine plasma concentration profile AUC₀₋ₜ of about 3000 to 4000 ng·hr/ml, or about 3300 to 3800 ng·hr/ml,
a ketamine plasma concentration profile Cₘₐₓ of about 20 to 50 ng/ml, or about 25 to 40 ng/ml,
a norketamine plasma concentration profile Cₘₐₓ of about 250 to 320 ng/ml, or about 260 to 300 ng/ml,
a ketamine plasma concentration profile Tₘₐₓ of about 1.5 to 2.5 hours, or about 1.5 to 2.4 hours,
a norketamine plasma concentration profile Tₘₐₓ of about 2 to 3 hours, or about 2 to 2.9 hours,
a ketamine plasma concentration profile t_{1/2} of about 7 to 8 hours, or about 7.2 to 7.8 hours,
a norketamine plasma concentration profile t_{1/2} of about 7.5 to 8.5 hours, or about 7.8 to 8.1 hours,
a plasma concentration profile with a ratio of AUC norketamine/ketamine of about 8 to 15, or about 9 to 13 (for example a ratio of AUC ketamine to AUC norketamine of between about 1:8 and 1:15, or about 1:9 and 1:13),
a plasma concentration profile with a ratio of AUC_{0-∞} norketamine/ketamine of about 8 to 15, or about 9 to 13 (for example a ratio of AUC_{0-∞} ketamine to AUC_{0-∞} norketamine of between about 1:8 and 1:15, or about 1:9 and 1:13),
a plasma concentration profile with a ratio of AUC₀₋ₜ norketamine/ketamine of about 8 to 15, or about 9 to 13 (for example a ratio of AUC₀₋ₜ ketamine to AUC₀₋ₜ norketamine of between about 1:8 and 1:15, or about 1:9 and 1:13),
a plasma concentration profile with a ratio of Cₘₐₓ norketamine/ketamine of about 6 to 10, or about 7 to 10 (for example a ratio of Cₘₐₓ ketamine to Cₘₐₓ norketamine of between about 1:6 and 1:10, or about 1:7 and 1:10).

### Stability

The extended release pharmaceutical composition showed very good stability. For example the extended release pharmaceutical composition may comprise about 90 to 110% of starting amount (or target amount) of the active agent at 6 months of storage at about 40° C. with about 75% relative humidity.

For example, the extended release pharmaceutical composition may comprise about 90 to 110% of starting amount (or target amount) of the active agent at 12 months, or 24 months of storage at about 25° C. with about 60% relative humidity.

For example, the amount of the active agent in the extended release pharmaceutical composition changed by less than or equal to about 10% following storage for 6 months at 40° C. at 75% relative humidity.

For example, the amount of the active agent changed by less than or equal to about 10% following storage for 12 months, or 24 months at 25° C. at 60% relative humidity.

The storage is preferably in a closed container. For example, with 50 tablets in 75 mL container.

The extended release pharmaceutical composition may be a tablet with a target amount of active agent (for example about 60 mg or 120 mg of ketamine). The tablet may contain about 90 to 110% of the target amount of ketamine when manufactured and/or during storage, for example, about 12 months, or 24 months of storage at about 25° C. with about 60% relative humidity, or about 6 months of storage at about 40° C. with about 75% relative humidity.

### Treatment

Depression is characterized by depressed mood, and markedly diminished interest or pleasure in activities. Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts. A variety of somatic symptoms may also be present. Though depressive feelings are common, especially after experiencing setbacks in life, depressive disorder is diagnosed only when the symptoms reach a threshold and last at least two weeks. Depression can vary in severity from mild to very severe. It is most often episodic but can be recurrent or chronic. Some people have only a single episode, with a full return to premorbid function. However, more than 50 percent of those who initially suffer a single major depressive episode eventually develop another.

Treatment-resistant depression includes unipolar depression that does not respond satisfactorily to one or more treatments that are optimally delivered. If the depression has not benefited from at least two adequate trials of medications from different classes in the current episode, clinically significant treatment resistance is present.

Any chronic, treatment-resistant depression may be treated by the methods described herein. Such depression may include, but is not limited to, any of: major depressive disorder, single episode, recurrent major depressive disorder-unipolar depression, seasonal affective disorder-winter depression, bipolar mood disorder-bipolar depression, mood disorder due to a general medical condition-with major depressive-like episode, and/or mood disorder due to a general medical condition-with depressive features, wherein those disorders are resistant to treatment in a given patient. Thus, any patient that presents one of those disorders and who has not responded to an adequate trial of one antidepressant in the current episode and has recurrent or chronic depressive symptoms for greater than 2 years can be treated by the methods of the invention. Manic Depressive illnesses are also described in Goodwin, et al. 2007.

Anxiety is a mood disorder characterized by nervousness, fear, apprehension, and worrying. Patients with anxiety disorders may report symptoms such as excessive worry, panic attacks, or avoidance of specific situations (e.g. social interactions, supermarkets). Treatment resistant anxiety (TRA; anxiety that has not resolved or improved despite adequate medication and psychotherapy) is relatively common, with approximately 30% of patients showing no response to treatment, and a further 30-40% of patients having a partial response (Brown 1996).

The disclosure provides a method of treating a patient for a condition selected from the group consisting of depression, anxiety, alcohol abuse disorder, and pain, comprising: selecting a patient in need of such treatment; and administering to the patient the extended release composition as disclosed herein, wherein the extended release composition treats the symptoms of said depression, anxiety, alcohol abuse disorder, and/or pain.

Depression includes treatment-resistant depression, major depressive disorder (MDD) (for example, without limitation, with acute suicidal ideation or behavior), and bipolar depression.

Anxiety includes treatment-resistant anxiety, and includes, but is not limited to, DSM-V Generalized Anxiety Disorder (GAD), Social Anxiety Disorder, Panic Disorder, Post-Traumatic Stress Disorder (PTSD), and/or Obsessive-Compulsive Disorder.

The invention will be illustrated in more detail with reference to the following Examples, but it should be understood that the present invention is not deemed to be limited thereto.

### EXAMPLES

### Comparative Example 1—Dissolution of PEO Composition

WO2019073408, WO2020194087 and WO2022064387 (Douglas Pharmaceuticals Limited) describe a dosage course for the treatment of treatment-resistant depression and treatment-resistant anxiety. The oral extended release tablets disclosed primarily included ketamine and PEO at dose strength of 30 mg, 60 mg, 120 mg, 180 mg and 240 mg. To further improve safety, extended release tablets are needed that do not include PEO, but provide the same or substantially the same critical pharmacokinetics as the dosage course shown to be effective in the BEDROC study which used R-107, an oral extended release tablet including PEO.

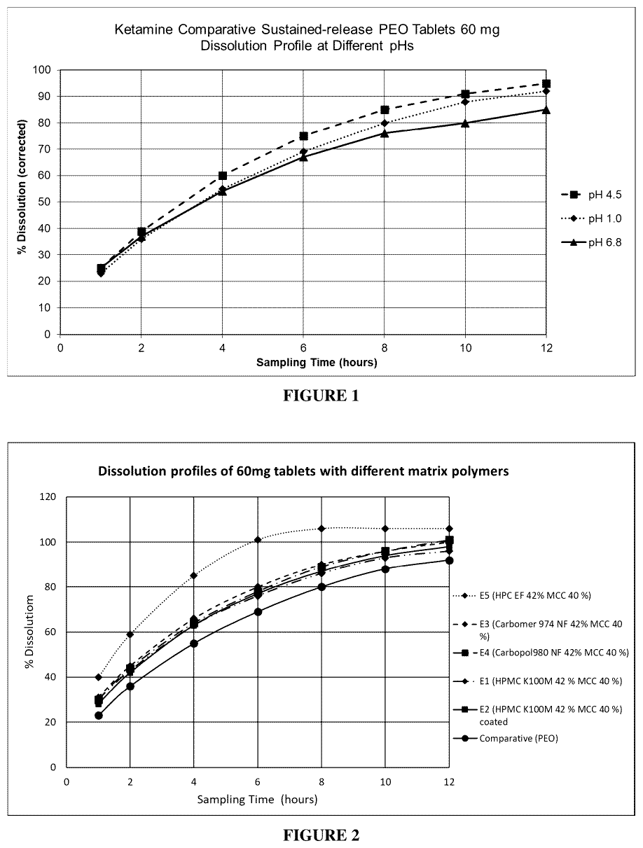

FIG. 1 of WO2019073408, WO2020194087 and WO2022064387 is a chart showing dissolution profiles of the 60 mg sustained release ketamine composition as a tablet with PEO at 3 different pHs. The dissolution results are reproduced below (Table 4 and FIG. 1) for comparison to the tablets disclosed herein.

TABLE 4
Dissolution data for 60 mg PEO sustained release ketamine
tablet at 3 different pHs, from WO2020194087 (FIG. 1)

| | Sampling time (hours) | | | | | | |
|---|---|---|---|---|---|---|---|
| | 1 | 2 | 4 | 6 | 8 | 10 | 12 |
| | % Dissolution (Corrected) | | | | | | |
| pH 6.8 | 25 | 37 | 54 | 67 | 76 | 80 | 85 |
| pH 4.5 | 25 | 39 | 60 | 75 | 85 | 91 | 95 |
| pH 1.0 | 23 | 36 | 55 | 69 | 80 | 88 | 92 |
| ±15% of pH 1 | 19.5-26.5 | 30.6-41.4 | 46.8-63 | 58.7-79.4 | 680.0-92.0 | 74.8-101.2 | 78.2-105.8 |
| dissolution | | | | | | | |

Comparison of dissolution profiles may provide an indication of likelihood of equivalent pharmacokinetics. Comparison of dissolution profiles is often used as a first in invitro indication of whether an oral tablet is likely to be considered generic (i.e. bioequivalent) to an innovator medicine. Difference factor (f1) and/or similarity factor (f2) comparisons of the dissolution curves are often calculated. Two dissolution profiles are considered similar when the f1 value is between 0 (identical) and 15 (15% difference) and/or the f2 value is between 50 and 100. For an indication of whether a curve is likely to be considered 15% different, the ±15% values for each dissolution sampling time point at pH 1 are also shown in Table 1.

### Example 2—Dissolution Testing Conditions

Tablets were made as detailed in the Examples below. The dissolution profiles of the tablets were measured using the conditions shown in Tables 5 and 6.

The dissolution tests were carried out on multiple samples (for example 2, 3 or 6 samples) and the average (mean) dissolution of the samples was calculated.

TABLE 5
Dissolution test conditions

| | |
|---|---|
| Apparatus: | Baskets, Apparatus 1 |
| Agitation speed: | 100 ± 4 rpm |
| Bath temperature: | 37.0 ± 0.5° C. |
| Volume (mL)/ | 900 mL/900 g |
| Mass (g) of | |
| dissolution | |
| medium per vessel: | |
| Sampling time: | 1, 2, 4, 6, 8, 10 and 12 hours (or as required) |
| Dissolution Media: | 0.1M Hydrochloric acid (for pH 1) |
| | Acetate Buffer (for pH 4.5) |
| | To prepare 1 L: Weigh 2.99 g of sodium acetate |
| | trihydrate, add 14 mL of 2 M acetic acid and dilute |
| | to 1000 mL with purified water. Adjust pH to 4.50 ± |
| | 0.05 with either 2M NaOH or 2M acetic acid as |
| | required. |
| | Phosphate Buffer (for pH 6.8) |
| | To prepare 1 L: Combine 250 mL of 0.2M |
| | potassium dihydrogen phosphate with 112 mL of |
| | 0.2M sodium hydroxide and dilute to 1000 mL with |
| | purified water. Adjust pH to 6.8 ± 0.05 with either |
| | 2M NaOH or phosphoric acid as required. |
| Sample Volume | 20 mL (withdraw for testing and time points and |
| | replace volume with dissolution medium) |

TABLE 6
UHPLC System Conditions

| | |
|---|---|
| System Description: | Waters Acquity H-Class UHPLC with UV-VWD |
| | or UV-PDA detector |
| Column: | C18, 2.5 μm, 2.1 × 150 mm (XBridge BEH |
| | Shield RP18 XP) (or equivalent) |
| Guard column: | C18, 2.5 μm, 2.1 × 5 mm (XBridge BEH |
| | Shield RP18 XP vanguard) (or equivalent) |
| Column | 40° C. |
| temperature: | |
| Detector wavelength: | 220 nm |
| Data collection rate: | 5 points/second |
| Flow rate: | 0.3 mL/min |
| Injection volume | 3 μL |
| Sampler temperature: | 20° C. |
| Inject Wash: | Mode: Post-inject |
| | Wash time: 6 seconds |
| Integration: | Peak area |
| Run time: | About 6 minutes |
| Retention time: | Ketamine: about 3 minutes |

The dissolution results were “corrected” to account for the samples being withdrawn from the dissolution vessel for testing and the same volume being replaced with new dissolution medium thus being diluted over the course of the testing. The raw measurements are “corrected” to account for the effect of the dilution.

### Example 3—Comparison of Matrix Polymers

Examples of tablets with different matrix polymers were made as shown in Table 7. The dissolution of the tablets was tested using the test conditions in Tables 5 (pH 1) and 6. The dissolution results are shown in Table 8 and graphically in FIG. 2.

Use of 42% w/w HPMC (for example E1 and E2) or Carbomer Homopolymer Type B (Carbopol® 974 NF) (for example E3) or Type C (Carbopol® 980 NF) (for example E4), with microcrystalline cellulose showed a good match to the dissolution of the PEO tablets.

Use of 42% w/w HPC EF as a matrix polymer (for example E5) showed a consistently faster dissolution profile than PEO, and was at 100% at only 6 hours. This composition is therefore not preferred. HPC EF has a lower viscosity than the HPMC or Carbomers used. HPC EF (Klucel) has a viscosity of 300-600 mPa·s (10% in water at 25° C.).

69.2 mg of ketamine HCl (17.3% w/w in the below compositions) is equivalent to 60 mg of free ketamine (15% w/w in the below compositions).

TABLE 7
Examples of tablets with different matrix polymers

| | Amount of ingredient - mg per tablet (% w/w) | | | | |
|---|---|---|---|---|---|
| Excipients | E1 | E2 | E3 | E4 | E5 |
| Ketamine HCl | 69.2 | 69.2 | 69.2 | 69.2 | 69.2 |
| (API) | (17.3) | (17.3) | (17.3) | (17.3) | (17.3) |
| HPMC K100M | 166.8 | 166.8 | — | — | — |
| (Matrix polymer) | (41.7) | (41.7) | | | |
| Carbopol ® 974 NF | — | — | 166.8 | — | — |
| (Carbomer | | | (41.7) | | |
| Homopolymer Type | | | | | |
| B USP NF) (Matrix | | | | | |
| polymer) | | | | | |
| Carbopol ® 980 NF | | — | | 166.8 | — |
| (Carbomer | | | | (41.7) | |
| Homopolymer Type | | | | | |
| C) (Matrix polymer) | | | | | |
| HPC EF (Matrix | | — | | | 166.8 |
| polymer) | | | | | (41.7) |
| MCC102 (permeability- | 160.0 | 160.0 | 160.0 | 160.0 | 160.0 |
| modifying filler) | (40.0) | (40.0) | (40.0) | (40.0) | (40.0) |
| Magnesium Stearate | 4.0 | 4.0 | 4.0 | 4.0 | 4.0 |
| (Lubricant) | (1.0) | (1.0) | (1.0) | (1.0) | (1.0) |
| Total: | 400 | 400 | 400 | 400 | 400 |
| | (100) | (100) | (100) | (100) | (100) |
| Opadry ® White Y-1- | — | 416 | — | — | — |
| 7000 (coating) | | (104) | | | |

TABLE 8
Dissolution results - pH 1

| | Sampling time (hours) | | | | | | |
|---|---|---|---|---|---|---|---|
| | 1 | 2 | 4 | 6 | 8 | 10 | 12 |
| | % Dissolution (Corrected) | | | | | | |
| E1 (avg of n = 6) | 30 | 43 | 63 | 76 | 86 | 93 | 96 |
| E2 (avg of n = 6) | 28 | 42 | 63 | 77 | 87 | 94 | 98 |
| E3 (avg of n = 2) | 31 | 45 | 66 | 80 | 90 | 96 | 100 |
| E4 (avg of n = 2) | 30 | 44 | 64 | 78 | 89 | 96 | 101 |
| E5 (avg of n = 2) | 40 | 59 | 85 | 101 | 106 | 106 | 106 |

“Avg of n = X” refers to average of “X” number of vessels tested.

The extended release tablets were made according to the following method:
1. Sieve through a 600 μm screen into a plastic bag to create a powder mixture:
   Matrix polymer (e.g. HPMC K100M)
   Ketamine (e.g. ketamine HCl)
   Filler (e.g. MCC 102)
2. Blend powder mixture using a V-cone blender for about 30 minutes until substantially homogeneous.
3. Sieve the lubricant (e.g. magnesium stearate) through a 600 μm screen into the mixture powder.
4. Blend using a V-cone blender for about 3 minutes until substantially homogeneous.
5. Directly compress the blend into tablets using the following settings:
   For 400 mg tablets:
   Compression machine: MINPRESS-II or Fette 1200i
   Punches: Oval Shape Tooling
   Aim tablet mass and range: 400.0 mg (380.0-420.0 mg)
   Aim tablet volume: 0.53 mL
   Aim Tablet Hardness: 120 to 250 Newtons or 100 to 250 Newtons
   Compression Force: Around 1 to 4 tons (about 10 to 40 kN)
   For 800 mg tablets:
   Compression machine: MINPRESS-II
   Punches: Capsule Shape Tooling
   Aim tablet mass and range: 800 mg (785-815 mg)
   Aim tablet volume: 1.06 mL
   Compression Force: Around 11 kN

Where the extended release tablets were coated, the following method was used:
1. Add purified water (e.g. 1125 g) to a stainless-steel vessel. Mix with an overhead stirrer.
2. Slowly add Opadry® White Y-1-7000 (e.g. 75 g) into the purified water while mixing with an overhead stirrer
3. Mix the coating solution for 45 minutes until the solution is free of lumps, taking care to prevent aeration of the solution.
4. Sieve the solution after mixing through <400 micron mesh.
5. Coat the tablets with the Opadry® solution:
   Spray coat with target coating bed temperature: 40-45° C.
   Aim weight gain: 4.0%.
   Acceptable weight gain: 3.0-5.0%

### Example 4—Comparison of Permeability-Modifying Fillers

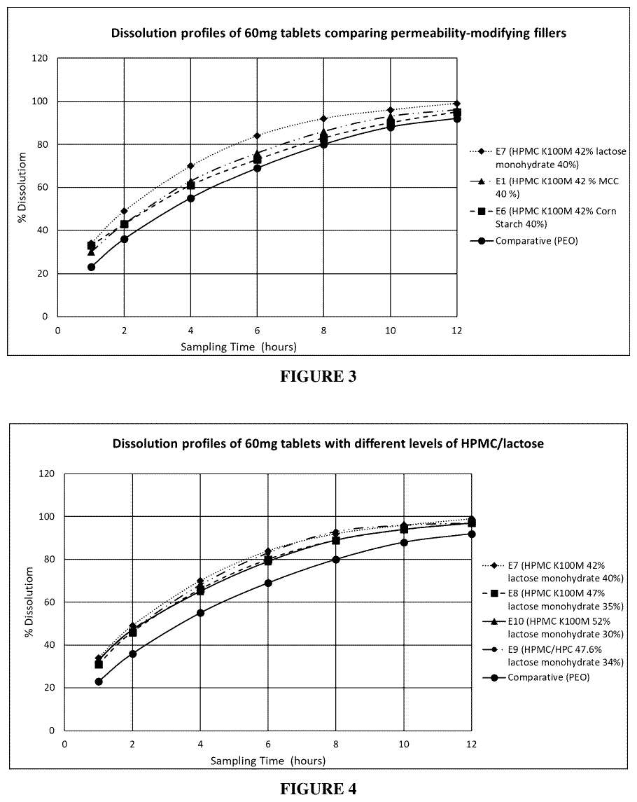

Tablets with different permeability-modifying fillers were made as shown in Table 9. The dissolution of the tablets was tested using the test conditions in Table 5 (pH 1) and 6. The dissolution results are shown in Table 10 and graphically in FIGS. 3 and 4.

As can be seen from the dissolution results in Table 10 and FIG. 3, use of 40% w/w corn starch (with 42% w/w HPMC) (for example E6) or 40% MCC (with 42% w/w HPMC) (for example E1) gave a good dissolution profile match to PEO.

Use of 40% w/w lactose monohydrate as a permeability-modifying filler showed a consistently faster dissolution profile (for example E7), so provided a less good match to the dissolution profile of PEO. Lactose monohydrate is a water soluble permeability-modifying filler. However, as shown in FIG. 4, reducing the amount of lactose monohydrate (to 35 and 30% w/w) (for example E8 and E10) and increasing the amount of HPMC by a corresponding amount (to 46.7 and 51.7% w/w) reduced the rate of dissolution, improving the match to PEO.

Where a combination of HPMC K100M (23.8% w/w) and HPC EF (23.8% w/w) (total of 47.6 5 w/w matrix polymer) with 34% w/w of lactose monohydrate (for example E9) was used the dissolution profile gave a similar match to using HPMC K100M as the sole polymer matrix. It is believed the addition of HPMC K100M increased the overall viscosity of the mixture of polymers.

TABLE 9
Examples of tablets with different permeability-modifying fillers

| | Amount of ingredient - mg per tablet (% w/w) | | | | | |
|---|---|---|---|---|---|---|
| Excipients | E1 | E6 | E7 | E8 | E9 | E10 |
| Ketamine HCl | 69.2 | 69.2 | 69.2 | 69.2 | 69.2 | 69.2 |
| | (17.3) | (17.3) | (17.3) | (17.3) | (17.3) | (17.3) |
| HPMC K100M | 166.8 | 166.8 | 166.8 | 186.8 | 95.2 | 206.8 |
| | (41.7) | (41.7) | (41.7) | (46.7) | (23.8) | (51.7) |
| HPC EF | — | — | — | — | 95.2 | — |
| | | | | | (23.8) | |
| MCC102 | 160.0 | — | — | — | — | — |
| | (40.0) | | | | | |
| Corn Starch | — | 160.0 | — | — | — | — |
| | | (40.0) | | | | |
| Lactose | — | — | 160.0 | 140.0 | 136.4 | 120.0 |
| monohydrate | | | (40.0) | (35.0) | (34.1) | (30.0) |
| Magnesium | 4.0 | 4.0 | 4.0 | 4.0 | 4.0 | 4.0 |
| Stearate | (1.0) | (1.0) | (1.0) | (1.0) | (1.0) | (1.0) |
| Total: | 400 | 400 | 400 | 400 | 400 | 400 |
| | (100) | (100) | (100) | (100) | (100) | (100) |

TABLE 10
Dissolution results - pH 1

Sampling time (hours)

| | | | | | | |
|---|---|---|---|---|---|---|
| 1 | 2 | 4 | 6 | 8 | 10 | 12 |

% Dissolution (Corrected)

| | | | | | | | |
|---|---|---|---|---|---|---|---|
| E1 | 30 | 44 | 64 | 78 | 89 | 95 | 100 |
| (avg of n = 6) | | | | | | | |
| E6 | 33 | 43 | 61 | 73 | 83 | 90 | 95 |
| (avg of n = 6) | | | | | | | |
| E7 | 34 | 49 | 70 | 84 | 92 | 96 | 99 |
| (avg of n = 6) | | | | | | | |
| E8 | 31 | 46 | 66 | 80 | 89 | 94 | 97 |
| (avg of n = 6) | | | | | | | |
| E9 | 33 | 47 | 68 | 83 | 93 | 96 | 97 |
| (avg of n = 6) | | | | | | | |
| E10 | 33 | 47 | 65 | 79 | 89 | 94 | 97 |
| (avg of n = 6) | | | | | | | |

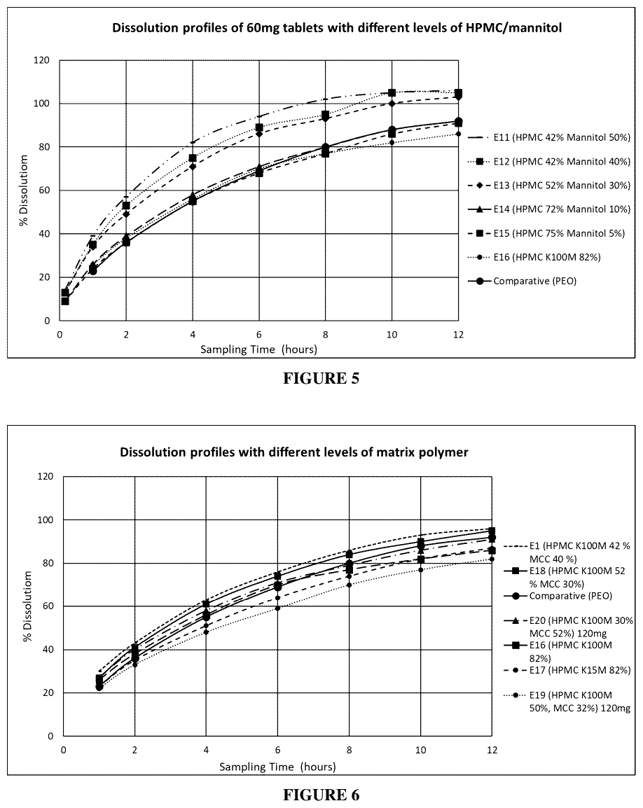

Another example of a water soluble permeability-modifying filler is mannitol. Tablets with varying amounts of mannitol were tested (50 to 0% w/w) as shown in Table 11. The dissolution of the tablets was tested using the test conditions in Table 5 (pH 1) and 6. The dissolution testing results are shown in Table 12 and graphically in FIG. 5.

TABLE 11
Examples of tablets with mannitol as
the permeability-modifying filler

| | Amount of ingredient - mg per tablet (% w/w) | | | | | |
|---|---|---|---|---|---|---|
| Excipients | E11 | E12 | E13 | E14 | E15 | E16 |
| Ketamine HCl | 69.2 | 69.2 | 69.2 | 69.2 | 69.2 | 69.2 |
| | (17.3) | (17.3) | (17.3) | (17.3) | (17.3) | (17.3) |
| HPMC | 126.8 | 166.8 | 206.8 | 286.8 | 306.8 | 326.8 |
| K100M | (31.7) | (41.7) | (51.7) | (71.7) | (76.7) | (81.7) |
| Mannitol | 200.0 | 160.0 | 120.0 | 40.0 | 20.0 | — |
| | (50.0) | (40.0) | (30.0) | (10.0) | (5.0) | |
| Magnesium | 4.0 | 4.0 | 4.0 | 4.0 | 4.0 | 4.0 |
| Stearate | (1.0) | (1.0) | (1.0) | (1.0) | (1.0) | (1.0) |
| Total: | 400 | 400 | 400 | 400 | 400 | 400 |
| | (100) | (100) | (100) | (100) | (100) | (100) |

TABLE 12
Dissolution results of compositions with mannitol - pH 1

| | Sampling time (hours) | | | | | | | |
|---|---|---|---|---|---|---|---|---|
| | 0.16 | 1 | 2 | 4 | 6 | 8 | 10 | 12 |
| | % Dissolution (Corrected) | | | | | | | |
| E11 | 14 | 39 | 57 | 82 | 94 | 102 | 105 | 106 |
| (avg of n = 6) | | | | | | | | |
| E12 | 13 | 35 | 53 | 75 | 89 | 95 | 105 | 105 |
| (avg of n = 6) | | | | | | | | |
| E13 | 13 | 34 | 49 | 71 | 86 | 93 | 100 | 103 |
| (avg of n = 6) | | | | | | | | |
| E14 | 9 | 26 | 39 | 58 | 71 | 80 | 88 | 92 |
| (avg of n = 3) | | | | | | | | |
| E15 | 9 | 24 | 36 | 55 | 68 | 77 | 86 | 91 |
| (avg of n = 3) | | | | | | | | |
| E16 | | 26 | 38 | 56 | 70 | 77 | 82 | 86 |
| (avg of n = 2) | | | | | | | | |

The tablet with only HPMC K100M (no permeability-modifying filler/mannitol) (for example E16) had a slow tail on the dissolution so is a less preferred match to PEO, as it may take a long time for dissolution to go to completion.

The tablets with 5-10% w/w mannitol (particularly 10% w/w mannitol) (for example E14 and E15) gave a good match to the PEO dissolution.

The tablets with 30-50% w/w mannitol (for example E13, E12 and E11) gave dissolution profiles which were a poor match to the PEO dissolution.

### Example 5—Dissolution at Different Dose Levels

The dissolution profile at higher dosing levels was tested (e.g. multiple tablets tested together) using tablets with the same composition as E1. The dissolution of the tablets was tested using the test conditions in Table 5 (pH 1) and 6. The results are shown in Table 13.

The dissolution profiles are substantially the same whether there was 1 tablet (60 mg), 2 tablets (120 mg) or 3 tablets (180 mg) tested at a time (i.e. 1, 2, or 3 tablets in the dissolution vessel). Therefore, a dose of 60 mg, 120 mg, and 180 mg shows substantially the same dissolution profile.

TABLE 13
Dissolution results of multiple tablets tested at the same time

| | Sampling time (hours) | | | | | | | |
|---|---|---|---|---|---|---|---|---|
| | 0.16 | 1 | 2 | 4 | 6 | 8 | 10 | 12 |
| | % Dissolution (Corrected) | | | | | | | |
| 60 mg tablet | 10 | 28 | 42 | 61 | 75 | 84 | 92 | 96 |
| (avg of n = 6) | | | | | | | | |
| 120 mg (2 × 60 | 10 | 28 | 41 | 59 | 72 | 82 | 89 | 94 |
| mg tablet) | | | | | | | | |
| (avg of n = 6) | | | | | | | | |
| 180 mg (3 × 60 | 9 | 26 | 40 | 58 | 70 | 81 | 88 | 94 |
| mg tablet) | | | | | | | | |
| (avg of n = 6) | | | | | | | | |

### Example 6—Amount of Matrix Polymer

Compositions with different amounts of matrix and/or formed into different size tablets were made as shown in Table 14. The dissolution of the tablets was tested using the test conditions in Table 5 (pH 1) and 6. The dissolution results are shown in Table 15 and graphically in FIG. 6.

Use of 82% w/w HPMC K100M (matrix polymer) (for example E16) with no permeability-modifying filler gave a slow tail of dissolution so is a less preferred match to PEO, as the dissolution may not go to completion for a long time. Use of 82% w/w HPMC K15M (matrix polymer) (for example E17) gave a consistently slower dissolution, so again is less preferred match to PEO.

Use of 52% and 42% w/w HPMC K100M (matrix polymer) with MCC (permeability-modifying filler) (for example E8 and E1) in a 400 mg tablet give a slightly faster but good match to the dissolution of PEO.

Use of 50% w/w HMPC with MCC in an 800 mg tablet (120 mg free ketamine) (for example E19) gave the slowest dissolution, so is less preferred. However, the speed of the dissolution in the larger tablet could be increased by lowering the amount of HPMC. The use of 30% w/w HPMC with MCC in an 800 mg tablet (for example E20) gave a good match to PEO.

As noted previously, 69.2 mg of ketamine HCl is equivalent to 60 mg of free ketamine. 138.4 mg of ketamine HCl is equivalent to 120 mg of free ketamine.

TABLE 14
Examples of tablets with different amount
of matrix polymer and different sizes

| | Amount of ingredient - mg per tablet (% w/w) | | | | | |
|---|---|---|---|---|---|---|
| Excipients | E16 | E17 | E18 | E1 | E19 | E20 |
| Ketamine | 69.2 | 69.2 | 69.2 | 69.2 | 138.4 | 138.4 |
| HCl | (17.3) | (17.3) | (17.3) | (17.3) | (17.3) | (17.3) |
| HPMC | 326.8 | — | 206.8 | 166.8 | 400.0 | 240.0 |
| K100M | (81.7) | | (51.7) | (41.7) | (50.0) | (30.0) |
| HPMC | — | 326.8 | — | — | — | — |
| K15M | | (81.7) | | | | |
| MCC102 | — | — | 120.0 | 160.0 | 258.4 | 414.4 |
| | | | (30) | (40) | (32.3) | (51.8) |
| Aerosil | — | — | — | — | — | 4.0 |
| (silicon | | | | | | (0.5) |
| dioxide) | | | | | | |
| Magnesium | 4.0 | 4.0 | 4.0 | 4.0 | 3.2 | 3.2 |
| Stearate | (1.0) | (1.0) | (1.0) | (1.0) | (0.4) | (0.4) |
| Total: | 400 | 400 | 400 | 400 | 800 | 800 |
| | (100) | (100) | (100) | (100) | (100) | (100) |

TABLE 15
Dissolution results - pH 1

Sampling time (hours)

| | | | | | | |
|---|---|---|---|---|---|---|
| 1 | 2 | 4 | 6 | 8 | 10 | 12 |

% Dissolution (Corrected)

| | | | | | | | |
|---|---|---|---|---|---|---|---|
| E16 (avg of | 26 | 38 | 56 | 70 | 77 | 82 | 86 |
| n = 2) | | | | | | | |
| E17 (avg of | 23 | 35 | 51 | 64 | 74 | 82 | 87 |
| n = 3) | | | | | | | |
| E18 (avg of | 27 | 41 | 61 | 74 | 84 | 90 | 95 |
| n = 3) | | | | | | | |
| E1 (avg of | 30 | 43 | 63 | 76 | 86 | 93 | 96 |
| n = 6) | | | | | | | |
| E19 (avg of | 22 | 33 | 48 | 60 | 70 | 77 | 82 |
| n = 3) | | | | | | | |
| E20 (avg of | 27 | 40 | 58 | 71 | 79 | 86 | 91 |
| n = 3) | | | | | | | |

### Example 7—Stability

The stability of tablets made according to composition E2 (including a coating of Opadry® White Y-1-7000) were tested.

The tablets were packaged for stability testing with 50 tablets per bottle in 75 mL HDPE white bottles with 38 mm cap made of polypropylene, white color, designed as a child resistant closure including a foil wad which acts as a tamper evident seal (CAP PP WHT CRC FOIL) and stored at 40° C. with 75% relative humidity (accelerated storage conditions) or at 25° C. with 60% relative humidity (long term storage conditions).

Results are shown in Tables 16A, 16B, and 17 below.

The tablets showed good stability when stored at 40° C. with 75% relative humidity over 6 months. The related substances (impurities) remained low and within specification. The assay (amount of active) remained high and within specification. The average dissolution remained consistent and within specification. The tablets also showed good long term stability when stored at 25° C. with 60% relative humidity. The total impurities remained below 0.2% (within specification) over 24 months. The assay remained within specification (90 to 110%) over 24 months. The average dissolution remained consistent and within specification over 24 months.

TABLE 16A
Stability testing results of coated E2 stored at
40° C. with 75% relative humidity (months 0 to 2)

| | | Months | | |
|---|---|---|---|---|
| Test | Specification | 0 | 1 | 2 |
| Average mass (mg) | — | 411 | 412 | 420 |

Related Substances (%)

| | | | | |
|---|---|---|---|---|
| Ketamine Related | NMT 0.2 | <0.04 | <0.04 | <0.04 |
| Compound A (%) | | | | |
| Ketamine Related | NMT 0.2 | <0.04 | <0.04 | <0.04 |
| Compound B (%) | | | | |
| Ketamine Related | NMT 0.2 | <0.04 | <0.04 | <0.04 |
| Compound C (%) | | | | |
| Unspecified | NMT 0.2 | <0.04 (0.33) | <0.04 (0.33) | <0.04 (0.33) |
| Impurities (%) (RRT) | | <0.04 (0.86) | <0.04 (0.86) | <0.04 (0.86) |
| | | <0.04 (0.91) | <0.04 (0.91) | <0.04 (0.88) |
| | | <0.04 (2.35) | <0.04 (2.35) | 0.04 (2.42) |
| Total Unspecified | NMT 0.2 | <0.04 | <0.04 | <0.04 |
| Impurities (%) | | | | |

Assay, Ketamine (%)

| | | | | |
|---|---|---|---|---|
| % of the labelled | 90.0 to 110.0 | 99.6 | 99.1 | 98.1 |
| content | | | | |

Average % Dissolution (Corrected) (avg of n = 6) (pH 1)

| | | | | |
|---|---|---|---|---|
| 60 minutes | No more than | 28 | 27 | 28 |
| 120 minutes | 40% at 1 hour. | 42 | 40 | 42 |
| 240 minutes | Not less than | 63 | 59 | 63 |
| 360 minutes | 40% and no | 77 | 73 | 77 |
| 480 minutes | more than 75% | 87 | 83 | 87 |
| 600 minutes | at 4 hours. | 94 | 90 | 94 |
| 720 minutes | Not less than | 98 | 95 | 100 |
| | 75% at 12 | | | |
| | hours. | | | |

TABLE 16B
Stability testing results of coated E2 stored at
40° C. with 75% relative humidity (months 3 to 6)

| | | Months | |
|---|---|---|---|
| Test | Specification | 3 | 6 |

| | | |
|---|---|---|
| Average mass (mg) | 414 | 409 |

Related Substances (%)

| | | | |
|---|---|---|---|
| Ketamine Related | NMT 0.2 | <0.04 | <0.04 |
| Compound A (%) | | | |
| Ketamine Related | NMT 0.2 | <0.04 | <0.04 |
| Compound B (%) | | | |
| Ketamine Related | NMT 0.2 | <0.04 | <0.04 |
| Compound C (%) | | | |
| Unspecified | NMT 0.2 | 0.14 (0.54) | <0.04 |
| Impurities (%) (RRT) | | 0.07 (0.67) | |
| Total Unspecified | NMT 0.2 | 0.21 | <0.04 |
| Impurities (%) | | | |

Assay, Ketamine (%)

| | | | |
|---|---|---|---|
| % of the labelled | 90.0 to 110.0 | 96.6 | 99.7 |
| content | | | |

Average % Dissolution (Corrected) (avg of n = 6) (pH 1)

| | | | |
|---|---|---|---|
| 60 minutes | No more than | 28 | 28 |
| 120 minutes | 40% at 1 hour. | 42 | 43 |
| 240 minutes | Not less than | 62 | 64 |
| 360 minutes | 40% and no | 76 | 78 |
| 480 minutes | more than 75% | 86 | 88 |
| 600 minutes | at 4 hours. | 92 | 94 |
| 720 minutes | Not less than | 96 | 98 |
| | 75% at 12 | | |
| | hours. | | |

NMT = Not more than
RRT = relative retention time. Ratio of retention time to that of the main peak, which serves as an ID.

TABLE 17
Stability testing results of coated E2 stored at 25° C. with 60% relative humidity

| | | Months | | | |
|---|---|---|---|---|---|
| Test | Specification | 0 | 6 | 12 | 24 |
| Average mass (mg) | — | 411 | 411 | 412 | 413 |
| Related Substances (%) | | | | | |
| Ketamine Related | NMT 0.2 | <0.04 | <0.04 | <0.04 | <0.04 |
| Compound A (%) | | | | | |
| Ketamine Related | NMT 0.2 | <0.04 | <0.04 | <0.04 | <0.04 |
| Compound B (%) | | | | | |
| Ketamine Related | NMT 0.2 | <0.04 | <0.04 | <0.04 | <0.04 |
| Compound C (%) | | | | | |
| Unspecified | NMT 0.2 | <0.04 (0.33) | <0.04 | 0.05 (0.21) | <0.04 |
| Impurities (%) (RRT) | | <0.04 (0.86) | | | |
| | | <0.04 (0.91) | | | |
| | | <0.04 (2.35) | | | |
| Total Impurities (%) | NMT 0.2 | <0.04 | <0.04 | 0.05 | <0.04 |
| Assay, Ketamine (%) | | | | | |
| % of the labelled | 90.0 to 110.0 | 99.6 | 97.6 | 97.7 | 97.7 |
| content | | | | | |
| Average % Dissolution (Corrected) | | | | | |
| (avg of n = 6) (pH 1) | | | | | |
| 60 minutes | No more than | 28 | 28 | 28 | 28 |
| 120 minutes | 40% at 1 hour. | 42 | 43 | 42 | 42 |
| 240 minutes | Not less than | 63 | 64 | 62 | 62 |
| 360 minutes | 40% and no | 77 | 78 | 76 | 76 |
| 480 minutes | more than 75% | 87 | 89 | 85 | 85 |
| 600 minutes | at 4 hours. | 94 | 94 | 92 | 92 |
| 720 minutes | Not less than | 98 | 99 | 96 | 96 |
| | 75% at 12 | | | | |
| | hours. | | | | |

NMT = Not more than
RRT = relative retention time. Ratio of retention time to that of the main peak, which serves as an ID.

### Example 8—In Vivo Testing

A single center, single dose, blinded, balanced two-treatment, two-period, two-sequence, two-way crossover, randomized bioavailability study was conducted. Participants received the Test product (E2) or the Reference product (composition used in BEDROC study), under fasting conditions in each of the two treatment periods. Each participant was randomly assigned to one of the treatment sequences shown in Table 18. Thirty-four participants entered the study and received at least one dose of study medication. Thirty-two participants completed both study periods.

TABLE 18
Treatment sequences

| | Period 1 | Period 2 |
|---|---|---|
| Sequence 1 | Test | Reference |
| Sequence 2 | Reference | Test |

Sequence 1 (3×60 mg tablets—180 mg dose, Test or Reference) was administered with 240 ml of ambient-temperature water after an overnight fast of at least 10 hours. Participants did not receive any food until at least 4 hours post-dose. The tablets were swallowed whole.

Participants were confined to the Clinical Site from at least 12 hours prior to drug administration, until after the 24-hour post-dose blood draw. During each study period, venous blood (8 ml) was collected prior to dosing (0 hour sample) and at 0.5, 1, 1.5, 2, 2.5, 3, 3.5, 4, 4.5, 5, 6, 7, 8, 9, 12, 18, 24, 32, and 48 hours post-dose (i.e., 20 blood samples profile/participant). Participants returned to the clinical site for the collection of the blood samples at 32 and 48 hours post-dosing. A total of 20×8 ml blood samples were collected for each participant. Sequence 2 was administered in the same way following a 1 week washout period.

The 34 adults (18 male and 16 female) that participated in the study were healthy, non-smoking, with a mean (range) age of 25 (18-42) years, a mean (range) weight of 73.1 (52.6-118.7) kg, a mean (range) height of 1.73 (1.47-1.98) m and a mean (range) BMI of 24.3 (20.0-30.9) kg/m². There were 18 (52.94%) participants who were White, 10 (29.41%) participants who were Asian, 2 (5.88%) participants who were White/Other Pacific Islander, 1 (2.94%) participant who was Black, 1 (2.94%) participant who was White/Asian, 1 (2.94%) participant who was Other Pacific Islander/Asian and 1 (2.94%) participant who was White/Asian/Other Pacific Islander.

### Analytical Methods:

Plasma ketamine and norketamine concentrations were determined using validated, sensitive LCMSMS methods. The range of quantification for determination of ketamine in plasma was from 0.30 to 38.40 ng/ml. The LLOQ for ketamine in plasma was 0.30 ng/ml. The range of quantification for determination of norketamine in plasma was from 3.50 to 448.00 ng/ml. The LLOQ for norketamine in plasma was 3.50 ng/ml.

### Statistical Methods:

The arithmetic mean, standard deviation (S.D) and inter-participant CV were calculated for the plasma concentrations of the participants at each sampling time and each formulation (Test and Reference) for the pharmacokinetic parameters AUC_{0-∞}, AUC₀₋ₜ, Cₘₐₓ, t_{1/2} and Tₘₐₓ for ketamine and norketamine.

Cₘₐₓ was the maximum observed plasma concentration. AUC_{0-∞} was the area under the plasma concentration-time curve from time zero to infinity calculated as the sum of AUC₀₋ₜ plus the ratio of the last measurable plasma concentration to the elimination rate constant. AUC₀₋ₜ was the area under the plasma concentration-time curve from time zero up to the last quantifiable concentration-time point calculated using the trapezoidal rule. Tₘₐₓ was the time of the maximum observed plasma concentration. t_{1/2} was apparent first-order plasma terminal elimination half-life calculated as 0.693/Kₑₗ. Kₑₗ is apparent first-order terminal elimination rate constant. Kₑₗ was calculated from a semi-log plot of the plasma concentration versus time curve. The parameter was calculated by linear least-squares regression analysis using at least four non-zero plasma concentrations from the terminal log-linear phase.

Analysis of variance (ANOVA) was performed for ketamine and norketamine in plasma on the log transformed AUC_{0-∞}, AUC₀₋ₜ and Cₘₐₓ and on the untransformed AUC_{0-∞}, AUC₀₋ₜ, Cmax, Tₘₐₓ, and t_{1/2}. The ANOVA model included period, sequence, participant within sequence, period and treatment as factors used for bioequivalence analysis. The statistical software used was SAS®, Version 6.0.

The ratio of the geometric means and the 90% confidence intervals were calculated based on the difference of the log-transformed AUC_{0-∞}, AUC₀₋ₜ and Cₘₐₓ between the Test and Reference products. Bioequivalence would be concluded if the 90% confidence intervals of AUC_{0-∞}, AUC₀₋ₜ and Cₘₐₓ for ketamine and norketamine in plasma were within 80.00% to 125.00%.

Relative bioavailability was calculated using the following formulae:

$$( ( AU {C}_{0 - t ( T )} / {Dose}_{( T )} ) / ( AU {C}_{0 - t ( R )} / {Dose}_{( R )} ) ) * 100 \% , and$$

$$( ( {C}_{max ( T )} / {Dose}_{( T )} ) / ( {C}_{max ( R )} / {Dose}_{( R )} ) ) * 100 \%$$

### Pharmacokinetic Results:

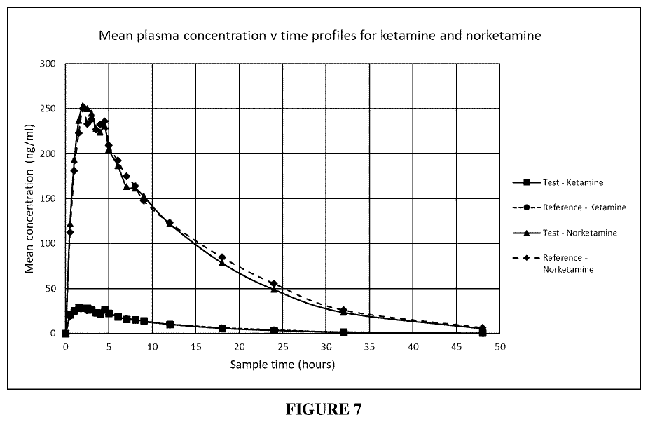

The mean and standard deviation (S.D) ketamine and norketamine plasma concentration for each sampling time are listed in Tables 19 and 20. Mean ketamine and norketamine plasma concentrations versus time are shown graphically in FIG. 7.

TABLE 19
Mean and standard deviation (S.D.) plasma ketamine
concentration data vs sampling time

| | Test Treatment (3 × 60 | | Reference Treatment (3 × 60 | |
|---|---|---|---|---|
| | mg ketamine tablets) | | mg ketamine tablets) | |
| Sample | Ketamine mean | | Ketamine mean | |
| time | concentration | | concentration | |
| (hrs) | (ng/ml) | S.D | (ng/ml) | S.D |
| 0 | 0.00 | 0.00 | 0.00 | 0.00 |
| 0.5 | 20.89 | 13.05 | 18.95 | 11.52 |
| 1 | 25.52 | 14.45 | 23.62 | 11.97 |
| 1.5 | 29.25 | 14.15 | 27.24 | 13.81 |
| 2 | 28.26 | 13.78 | 28.45 | 14.18 |
| 2.5 | 28.08 | 14.18 | 24.99 | 12.42 |
| 3 | 26.54 | 14.31 | 25.36 | 13.52 |
| 3.5 | 23.14 | 11.04 | 23.77 | 13.03 |
| 4 | 22.04 | 11.11 | 23.59 | 13.23 |
| 4.5 | 26.58 | 17.40 | 27.47 | 16.45 |
| 5 | 22.58 | 15.70 | 22.53 | 13.49 |
| 6 | 18.82 | 11.31 | 19.61 | 11.93 |
| 7 | 15.95 | 9.63 | 17.15 | 11.98 |
| 8 | 15.29 | 9.26 | 15.45 | 10.19 |
| 9 | 13.90 | 9.05 | 13.40 | 7.72 |
| 12 | 10.30 | 6.08 | 10.56 | 5.88 |
| 18 | 5.88 | 3.57 | 6.57 | 3.84 |
| 24 | 3.60 | 2.26 | 4.31 | 2.74 |
| 32 | 1.69 | 1.49 | 1.74 | 1.22 |
| 48 | 0.41 | 0.43 | 0.43 | 0.44 |

TABLE 20
Mean and standard deviation (S.D.) plasma norketamine
concentration data vs sampling time

| | Test Treatment (3 × 60 | | Reference Treatment (3 × 60 | |
|---|---|---|---|---|
| | mg ketamine tablets) | | mg ketamine tablets) | |
| Sample | Norketamine mean | | Norketamine mean | |
| time | concentration | | concentration | |
| (hrs) | (ng/ml) | S.D | (ng/ml) | S.D |
| 0 | 0.00 | 0.00 | 0.00 | 0.00 |
| 0.5 | 122.04 | 60.75 | 112.54 | 51.64 |
| 1 | 193.26 | 65.37 | 180.94 | 53.35 |
| 1.5 | 237.17 | 77.94 | 222.94 | 62.43 |
| 2 | 253.33 | 78.23 | 249.80 | 70.93 |
| 2.5 | 249.87 | 66.33 | 233.11 | 57.49 |
| 3 | 244.92 | 63.94 | 238.33 | 67.12 |
| 3.5 | 227.17 | 63.24 | 227.02 | 63.37 |
| 4 | 223.91 | 67.42 | 232.62 | 77.15 |
| 4.5 | 231.02 | 83.69 | 236.04 | 78.33 |
| 5 | 204.63 | 73.33 | 209.07 | 73.31 |
| 6 | 186.84 | 64.20 | 192.17 | 72.23 |
| 7 | 163.38 | 52.15 | 174.95 | 68.75 |
| 8 | 161.66 | 57.07 | 164.08 | 61.45 |
| 9 | 152.63 | 55.61 | 147.47 | 50.25 |
| 12 | 121.96 | 42.60 | 123.08 | 42.77 |
| 18 | 78.69 | 33.57 | 85.02 | 35.59 |
| 24 | 49.06 | 21.76 | 55.71 | 24.85 |
| 32 | 23.56 | 17.43 | 26.23 | 16.08 |
| 48 | 5.56 | 5.46 | 6.51 | 6.01 |

The pharmacokinetic parameters for ketamine in plasma from the Test and the Reference dosing are summarized in Tables 21 and 22 (untransformed and geometric data respectively).

TABLE 21
Pharmacokinetic parameters for ketamine (untransformed data)

| | | Reference |
|---|---|---|
| | Test Treatment (A): | Treatment (B): |
| | 3 × 60 mg ketamine | 3 × 60 mg ketamine |
| Pharmacokinetic | tablets (n = 32) mean ± | tablets (n = 32) mean ± |
| Parameter | S.D (range) | S.D (range) |
| AUC_{0-∞} (ng · | 347.31 ± 181.37 | 355.08 ± 183.46 |
| hr/ml) | (49.18-794.97) | (64.21-770.71) |
| AUC₀₋ₜ (ng · | 338.53 ± 178.51 | 347.52 ± 180.94 |
| hr/ml) | (46.77-774.45) | (60.08-742.39) |
| Cₘₐₓ (ng/ml) | 37.45 ± 17.64 | 33.94 ± 16.62 |
| | (4.59-72.19) | (5.51-73.33) |
| Tₘₐₓ (hr) | 1.91 ± 1.18 | 2.31 ± 1.40 |
| | (0.50-4.50) | (0.50-6.00) |
| t_{1/2} (hr) | 7.67 ± 1.65 | 7.55 ± 1.41 |
| | (4.71-11.03) | (4.50-11.16) |

TABLE 22
Pharmacokinetic parameters for ketamine (geometric data)

| | | Reference |
|---|---|---|
| | Test Treatment (A): | Treatment (B): |
| | 3 × 60 mg ketamine | 3 × 60 mg ketamine |
| Pharmacokinetic | tablets (n = 32) mean ± | tablets (n = 32) mean ± |
| Parameter | S.D^{a} (range) | S.D^{a} (range) |
| AUC_{0-∞} (ng · | 295.03 ± 188.81 | 304.06 ± 186.80 |
| hr/ml) | (49.18-794.97) | (64.21-770.71) |
| AUC₀₋ₜ (ng · | 286.27 ± 186.21 | 296.02 ± 186.17 |
| hr/ml) | (46.77-774.45) | (60.08-742.39) |
| Cₘₐₓ (ng/ml) | 31.60 ± 22.07 | 28.98 ± 18.76 |
| | (4.59-72.19) | (5.51-73.33) |
| Tₘₐₓ (hr) | 1.60 ± 0.99 | 1.96 ± 1.14 |
| | (0.50-4.50) | (0.50-6.00) |

^{a}Jackknife Standard Deviation

The pharmacokinetic parameters for norketamine in plasma from the Test and the Reference dosing are summarized in Tables 23 and 24 (untransformed and geometric data respectively).

TABLE 23
Pharmacokinetic parameters for norketamine (untransformed data)

| | | Reference |
|---|---|---|
| | Test Treatment (A): | Treatment (B): |
| | 3 × 60 mg ketamine | 3 × 60 mg ketamine |
| Pharmacokinetic | tablets (n = 32) (mean ± | tablets (n = 32) (mean ± |
| Parameter | S.D) (Range) | S.D) (Range) |
| AUC_{0-∞} (ng · | 3763.13 ± 1175.57 | 3867.06 ± 1251.75 |
| hr/ml) | (1583.26-6004.73) | (2009.49-6231.62) |
| AUC₀₋ₜ (ng · | 3626.28 ± 1143.98 | 3753.23 ± 1217.92 |
| hr/ml) | (1519.10-5684.02) | (1926.14-5984.06) |
| Cₘₐₓ (ng/ml) | 290.94 ± 77.12 | 281.53 ± 73.82 |
| | (155.65-434.21) | (168.34-440.69) |
| Tₘₐₓ (hr) | 2.45 ± 1.18 | 2.74 ± 1.20 |
| | (0.52-5.05) | (1.05-7.00) |
| t_{1/2} (hr) | 7.95 ± 1.77 | 7.91 ± 1.68 |
| | (4.58-11.28) | (4.99-12.10) |

TABLE 24
Pharmacokinetic parameters for norketamine (geometric data)

| | | Reference |
|---|---|---|
| | Test Treatment (A): | Treatment (B): |
| | 3 × 60 mg ketamine | 3 × 60 mg ketamine |
| Pharmacokinetic | tablets (n = 32) (mean ± | tablets (n = 32) (mean ± |
| Parameter | S.D^{a}) (Range) | S.D^{a}) (Range) |
| AUC_{0-∞} (ng · | 3570.58 ± 1219.02 | 3666.16 ± 1238.98 |
| hr/ml) | (1583.26-6004.73) | (2009.49-6231.62) |
| AUC₀₋ₜ (ng · | 3438.12 ± 1180.62 | 3556.08 ± 1211.03 |
| hr/ml) | (1519.10-5684.02) | (1926.14-5984.06) |
| Cₘₐₓ (ng/ml) | 280.64 ± 78.10 | 272.47 ± 70.63 |
| | (155.65-434.21) | (168.34-440.69) |
| Tₘₐₓ (hr) | 2.16 ± 1.17 | 2.52 ± 1.04 |
| | (0.52-5.05) | (1.05-7.00) |

^{a}Jackknife Standard Deviation

Table 25 shows the ratio of the AUC and Cₘₐₓ of norketamine to ketamine for the Test and Reference treatments. The ratios are extremely similar for AUC (0-∞ or 0-t) and very similar for Cₘₐₓ. While not wishing to be bound by theory, it is believed the amounts and/or ratios of norketamine and ketamine are important to the efficacy of the Reference product shown in the BEDROC study (Glue et al. “Extended-release ketamine tablets for treatment-resistant depression: a randomized placebo-controlled phase 2 trial”, Nature Medicine, Volume 30, July 2024, 2004-2009). It is therefore important for bioequivalence and/or efficacy that the Test Treatment shows substantially the same ratios. For comparison, an immediate release ketamine tablet (oral dosing) showed Cₘₐₓ ratio (Norketamine/Ketamine) of 4.3, ratio of AUC₀₋ₜ (Norketamine/Ketamine) of 7.5, ketamine Tₘₐₓ of 0.52 hours and norketamine Tₘₐₓ of 0.39 hours (Yanagihara, Y. et al. Plasma Concentration Profiles of Ketamine and Norketamine after Administration of Various Ketamine Preparations to Healthy Japanese Volunteers. Biopharm. Drug Dispos. 24: 37-43 (2003)).

TABLE 25
Ratio of pharmacokinetic parameters for norketamine/ketamine

| | | Reference |
|---|---|---|
| | Test Treatment (A): | Treatment (B): |
| | 3 × 60 mg ketamine | 3 × 60 mg ketamine |
| Pharmacokinetic | tablets (n = 32) (mean ± | tablets (n = 32) (mean ± |
| Parameter | S.D^{a}) (Range) | S.D^{a}) (Range) |
| Ratio of AUC_{0-∞} | 10.84 (untransformed | 10.89 (untransformed |
| Norketamine/ | data) | data) |
| Ketamine | 12.10 (geometric data) | 12.06 (geometric data) |
| Ratio of AUC₀₋ₜ | 10.71 (untransformed | 10.80 (untransformed |
| Norketamine/ | data) | data) |
| Ketamine | 12.01 (geometric data) | 12.01 (geometric data) |
| Ratio of Cₘₐₓ | 7.77 (untransformed | 8.29 (untransformed |
| Norketamine/ | data) | data) |
| Ketamine | 8.88 (geometric data) | 9.40 (geometric data) |

Tables 26 and 27 show the Least Squares Geometric Mean Ratio, 90% Confidence Intervals, Intra Participant CV and Relative Bioavailability for ketamine and norketamine in the comparison of 3×60 mg Test and 3×60 mg Reference.

TABLE 26
Comparison of the pharmacokinetic parameters for ketamine
for the 3 × 60 mg Test and Reference products

| | Least squares | 90% | Intra | Relative |
|---|---|---|---|---|
| | geometric | confidence | participant | Bioavailability |
| | mean ratio % | interval | CV % | % |
| AUC_{0-∞} | 97.03 | (92.54, 101.74) | 11.21 | — |
| AUC₀₋ₜ | 96.70 | (92.23, 101.39) | 11.19 | 97.41 |
| Cₘₐₓ | 109.05 | (102.97, 115.48) | 13.56 | 110.33 |

TABLE 27
Comparison of the pharmacokinetic parameters for norketamine
for the 3 × 60 mg Test and Reference products

| | Least squares | 90% | Intra | Relative |
|---|---|---|---|---|
| | geometric | confidence | participant | Bioavailability |
| | mean ratio % | interval | CV % | % |
| AUC_{0-∞} | 97.39 | (93.68, 101.25) | 9.18 | |
| AUC₀₋ₜ | 96.68 | (92.99, 100.53) | 9.20 | 96.62 |
| Cₘₐₓ | 103.00 | (99.08, 107.08) | 9.17 | 103.34 |

The 90% confidence intervals of AUC_{0-∞} (92.54,101.74), AUC₀₋ₜ (92.23,101.39) and Cₘₐₓ (102.97,115.48) for ketamine in plasma and AUC_{0-∞} (93.68,101.25), AUC₀₋ₜ (92.99,100.53) and Cₘₐₓ (99.08,107.08) for norketamine in plasma resulting from the comparison of the Test and the Reference products were within the accepted bioequivalence criteria limits of 80.00% and 125.00% for log₁₀ transformed data.

Relative bioavailability for ketamine and norketamine AUC₀₋ₜ were 97.41% and 96.62% respectively in plasma. Relative bioavailability for ketamine and norketamine Cₘₐₓ were 110.33% and 103.34%, respectively in plasma.

Based on the ketamine and norketamine pharmacokinetic results obtained, it was concluded that the Test 60 mg tablets were bioequivalent to the Reference 60 mg tablets when administered as a single 180 mg (3×60 mg tablets) dose to healthy, adult participants under fasting conditions. The Cₘₐₓ was a little higher for the Test Treatment than the Reference Treatment (while still being considered to be within the accepted bioequivalence criteria).

The Test 60 mg ketamine tablets and the Reference 60 mg ketamine tablets were both deemed to be well tolerated as all adverse events reported by the 34 participants were deemed to be minor or not related to administration of the study drug.

1. An extended release pharmaceutical composition comprising:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
a matrix polymer, and
a permeability-modifying filler.

2. The composition of claim 1, wherein the matrix comprises about 30 to 75% w/w of the matrix polymer and about 20 to 60% w/w of the permeability-modifying filler.

3. The composition of claim 1, wherein the permeability-modifying filler is slightly soluble, very slightly soluble, practically insoluble, or insoluble in water at about 20° C.

4. The composition of claim 1, wherein the composition comprises about 12 to 30% w/w of the active agent.

5. The composition of claim 1, wherein the pharmaceutical composition is a tablet comprising about 50 to 80 mg of ketamine.

6. The composition of claim 1, wherein the matrix polymer is selected from one or more of the group hydroxypropyl methylcellulose, a carbomer homopolymer, and hydroxypropylcellulose.

7. The composition of claim 1, wherein the ratio of the active agent to the matrix polymer is about 1:1.5 to 1:4.

8. The composition of claim 1, wherein the permeability-modifying filler is selected from one or more of microcrystalline cellulose, starch (for example, without limitation, corn starch, pea starch, potato starch, rice starch, tapioca starch, wheat starch, arrowroot starch), calcium carbonate, and dicalcium phosphate.

9. The composition of claim 1, wherein the composition exhibits an in vitro release with at least three of the following:
(a) about 25 to 35% of the active substance is released at about the 1 hour time point;
(b) about 36 to 50% of the active substance is released at about the 2 hour time point;
(c) about 53 to 73% of the active substance is released at about the 4 hour time point;
(d) about 64 to 88% of the active substance is released at about the 6 hour time point;
(e) about 73 to 99% of the active substance is released at about the 8 hour time point;
(f) about 79 to 100% of the active substance is released at about the 10 hour time point;
(g) about 81 to 100% of the active substance is released at about the 12 hour time point;
when tested according to the USP Apparatus 1 (basket) method at a rotation rate of 100 rpm in media at pH 1.0 at 37.0±0.5° C.

10. The composition of claim 1, wherein the composition exhibits an in vitro release of:
about 31 to 44 mg of ketamine at 4 hours, and
about 48 to 60 mg of ketamine at 12 hours
when tested according to the USP Apparatus 1 (basket) method at a rotation rate of 100 rpm in media at pH 1.0 at 37.0±0.5° C.,
wherein the extended release pharmaceutical composition is a tablet comprising about 60 mg of ketamine.

11. The composition of claim 1, wherein administration of the extended release pharmaceutical composition to a subject providing a dose of about 180 mg of ketamine results in a ketamine plasma concentration profile AUC of about 270 to 370 ng·hr/ml

12. The composition of claim 1, wherein administration of the extended release pharmaceutical composition to a subject providing a dose of about 180 mg of ketamine results in a norketamine plasma concentration profile AUC of about 3000 to 4000 ng·hr/ml.

13. The composition of claim 1, wherein administration of the extended release pharmaceutical composition to a subject providing a dose of about 180 mg of ketamine results in a ketamine plasma concentration profile Cₘₐₓ of about 20 to 50 ng/ml.

14. The composition of claim 1, wherein administration of the extended release pharmaceutical composition to a subject providing a dose of about 180 mg of ketamine results in a norketamine plasma concentration profile Cₘₐₓ of about 250 to 320 ng/ml.

15. The composition of claim 1, wherein administration of the extended release pharmaceutical composition to a subject providing a dose of about 180 mg of ketamine results in a plasma concentration profile with a ratio of AUC norketamine/ketamine of about 8 to 15.

16. The composition of claim 1, wherein the extended release pharmaceutical composition comprises about 90 to 110% of a starting amount of the active agent after 24 months of storage at 25° C. with 60% relative humidity.

17. The composition of claim 1, wherein the matrix comprises:
about 30 to 75% w/w of a matrix polymer,
about 20 to 60% w/w of a permeability-modifying filler,
wherein the permeability-modifying filler is slightly soluble, very slightly soluble, practically insoluble, or insoluble in water at about 20° C., and
wherein the pharmaceutical composition is a tablet with a mass of about 300 to 600 mg and/or the tablet comprises about 50 to 80 mg of ketamine.

18. The composition of claim 1, wherein the matrix comprises:
about 45 to 80% w/w of a matrix polymer, and
about 3 to 35% w/w of a permeability-modifying filler,
wherein the permeability-modifying filler is very soluble or freely soluble in water at about 20° C., and
wherein the pharmaceutical composition is a tablet with a mass of about 300 to 600 mg and/or comprises about 50 to 80 mg of ketamine.

19. The composition of claim 1, wherein the matrix comprises:
about 20 to 45% w/w of a matrix polymer, and
about 40 to 65% w/w of a permeability-modifying filler,
wherein the pharmaceutical composition is a tablet with a mass of about 700 to 1000 mg and/or comprises about 100 to 140 mg of ketamine, and wherein the permeability-modifying filler is slightly soluble, very slightly soluble, practically insoluble, or insoluble in water at about 20° C.

20. A method of preventing, treating and/or managing depression, anxiety, alcohol abuse disorder, and/or pain in a subject, the method comprising administering an extended release pharmaceutical composition, wherein the extended release pharmaceutical composition comprises:
a therapeutically effective amount of an active agent selected from the group consisting of ketamine, norketamine, pharmaceutically acceptable salts thereof, and combinations thereof,
wherein the active agent is incorporated into a sustained-release polymeric matrix, the matrix comprising:
a matrix polymer, and
a permeability-modifying filler.

21. A method of manufacturing an extended release pharmaceutical composition, the method comprising:
blending a therapeutically effective amount of an active agent selected from the group consisting of ketamine, norketamine, pharmaceutically acceptable salts thereof, and combinations thereof, with a matrix polymer and a permeability-modifying filler.
